(19) United States
(12) Patent Application Publication
(10) Pub. No.: US 2025/0375434 A1
(43) Pub. Date: Dec. 11, 2025

(54) SMALL MOLECULES AND METHODS FOR INDUCING CARDIOMYOCYTE PROLIFERATION

(71) Applicant: UNIVERSITY OF VIRGINIA PATENT FOUNDATION, Charlottesville, VA (US)

(72) Inventors: Jeffrey Saucerman, Charlottesville, VA (US); David L. Brautigan, Charlottesville, VA (US); Laura Woo, Charlottesville, VA (US); Kaitlyn Wintruba, Charlottesville, VA (US)

(21) Appl. No.: 19/232,709

(22) Filed: Jun. 9, 2025

Related U.S. Application Data
(60) Provisional application No. 63/657,690, filed on Jun. 7, 2024.
(60) Provisional application No. 63/695,276, filed on Sep. 16, 2024.

(51) Int. Cl. A61K 31/444; A61K 31/405; A61K 31/415; A61K 31/4545; A61K 31/4709; A61K 45/06; C12N 5/077

(52) U.S. Cl. CPC A61K 31/444; A61K 31/405; A61K 31/415; A61K 31/4545; A61K 31/4709; A61K 45/06; C12N 5/0657

(57) ABSTRACT

Methods for inducing proliferation of cardiomyocytes in vitro and in vivo are disclosed.

### CROSS-REFERENCE TO RELATED APPLICATION

This application claims benefit of priority to U.S. Provisional Patent Application Nos. 63/657,690, filed Jun. 7, 2024, and 63/695,276, filed Sep. 16, 2024. The contents of which are incorporated by reference in their entireties.

### STATEMENT REGARDING FEDERALLY SPONSORED RESEARCH

N/A

### BACKGROUND

Over 5 million people in the United States suffer from heart failure due to the limited ability to regenerate functional cardiac tissue. Most pathologies that lead to heart failure, including myocardial infarction (MI) and several cardiomyopathies, cause irreversible loss of cardiac muscle. As a result, current therapies can only slow or reverse limited aspects of cardiac dysfunction. Adult mice and humans exhibit a low ˜1% cardiomyocyte (CM) renewal rate with a small amount of increased CM proliferation after myocardial infarction or mechanical unloading. Therefore, therapeutic strategies for cardiac damage that stimulate proliferation of preexisting cardiomyocytes are of interest.

### SUMMARY

In a first aspect, provided herein is a method for inducing proliferation of a cardiomyocyte culture, the method comprising contacting the cardiomyocyte culture with a therapeutic agent selected

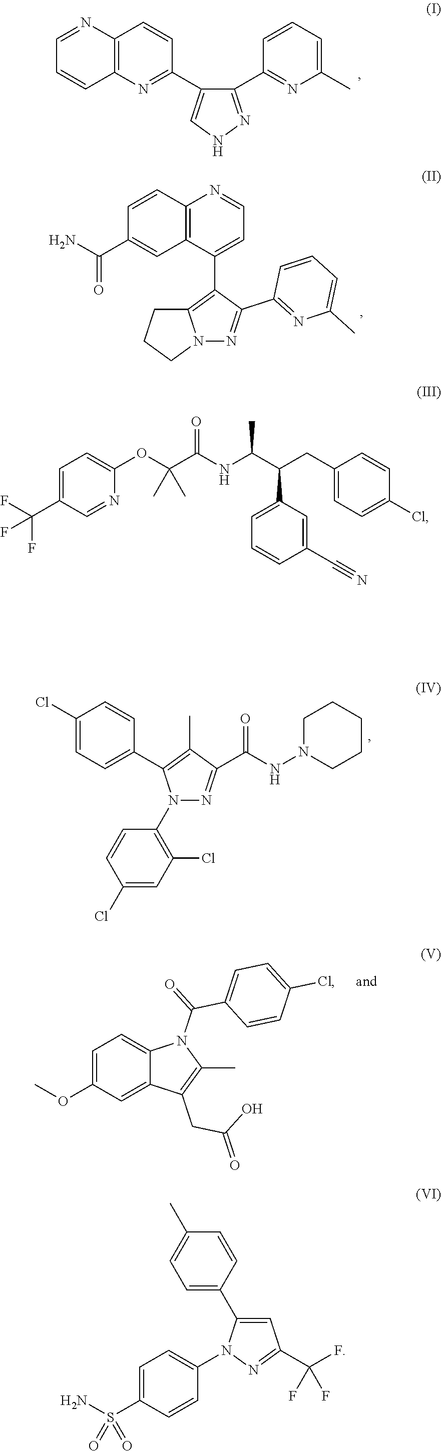

The therapeutic agent may be

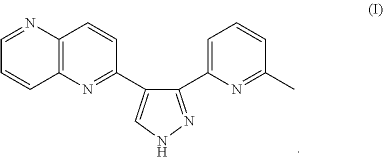 [SMILES: Cc1cccc(-c2n[nH]cc2-c2ccc3ncccc3n2)n1]

The therapeutic agent may be provided at a concentration of between about 0.1 and about 10 μM. The

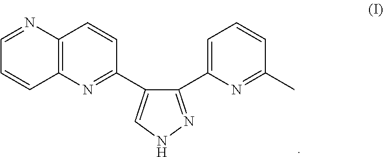 [SMILES: Cc1cccc(-c2n[nH]cc2-c2ccc3ncccc3n2)n1]

may be provided at a concentration of about 3 μM. The cardiomyocyte culture may comprise binucleated or polyploid cardiomyocytes, and contacting the culture with the therapeutic agent does not increase the amount of binucleated or polyploid cardiomyocytes. The cardiomyocyte culture may comprise primary cells or a cell line. The cell line may be the iCell cardiomyocyte cell line.

In another aspect, provided herein is a method for inducing expression of at least one of VEGFR2 (KDR) and ErbB2 expression in a cell, the method comprising contacting the cell with

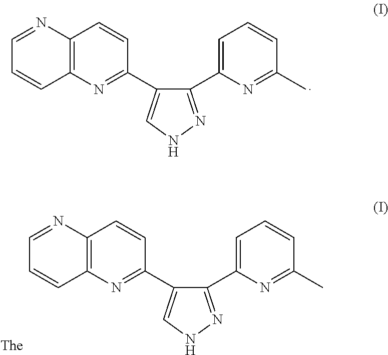 [SMILES: Cc1cccc(-c2n[nH]cc2-c2ccc3ncccc3n2)n1 ; Cc1cccc(-c2n[nH]cc2-c2ccc3ncccc3n2)n1]

may be provided at a concentration of between about 0.1 and about 10 μM. The

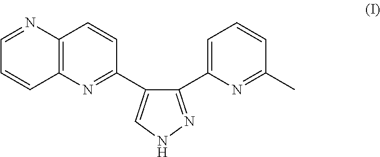 [SMILES: Cc1cccc(-c2n[nH]cc2-c2ccc3ncccc3n2)n1]

may be provided at a concentration of about 3 μM. The cell may be a cardiomyocyte.
In another aspect, provided herein is a method for treating heart damage in a subject in need thereof, the method comprising administering to the subject an effective amount of a therapeutic agent selected from

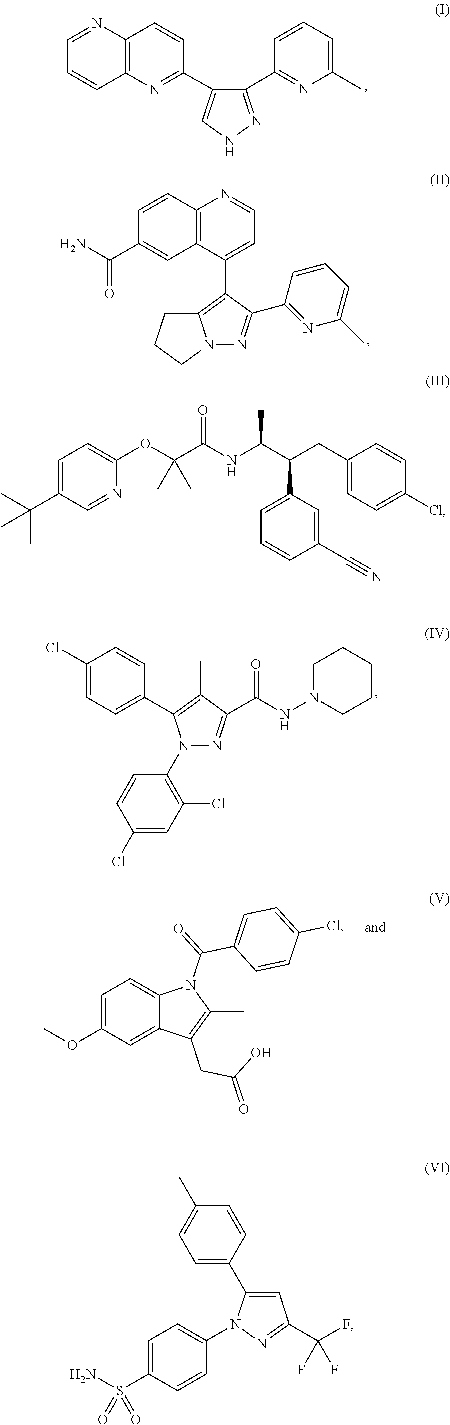

or a pharmaceutically acceptable salt thereof. The therapeutic agent may be

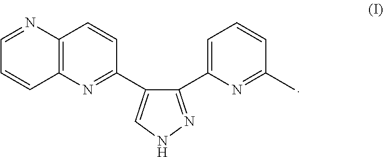 [SMILES: Cc1cccc(-c2n[nH]cc2-c2ccc3ncccc3n2)n1]

The heart damage may be a result of at least one of chemotherapy, a receptor tyrosine kinase inhibitor therapy, recreational drug use, heart disease, and a heart attack.

The method may further comprise administering an additional therapy selected from chemotherapy, a receptor tyrosine kinase inhibitor therapy, and a heart disease therapy. The additional therapy may be administered before, during or after the therapeutic agent. The subject may be a human.

In another aspect, provided herein is a method for treating a subject in need thereof, the method comprising administering to the subject an effective amount of a therapeutic agent selected

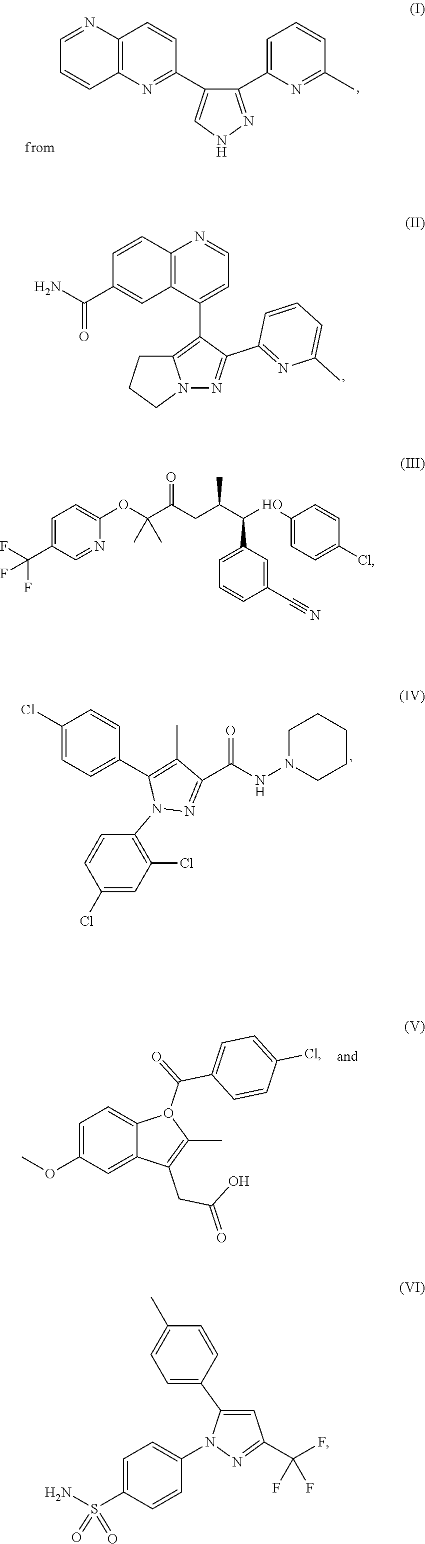

or a pharmaceutically acceptable salt thereof, wherein the subject is genetically predisposed to a heart defect or a heart condition. The subject may be genetically predisposed to a heart defect or a heart condition.

The disclosure is not an extensive overview of all contemplated features, steps, or advantages of the disclosed embodiments and is intended neither to identify key or critical elements of all aspects thereof nor to delineate the scope of any or all aspects of covered embodiments.

Aspects of the disclosure will become more fully understood upon a review of the drawings, and detailed description. Other aspects, features, and embodiments of the present disclosure will become apparent to those skilled in the art, upon reviewing the following (and attached) description of specific, example embodiments of the present disclosure in conjunction with the accompanying figures. While features of the present disclosure may be discussed relative to certain embodiments and figures, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1H. Compounds targeting distinct pathways induce proliferation in hiPSC-CMs with minimal multinucleation and nuclear polyploidy. A) Schematic of study. B) Representative images of 48 hr EdU assay measuring DNA synthesis rates of hiPSC-CMs treated with a combination of compound and 1 μM EdU. Cells were fixed and stained with DAPI (blue) and EdU (magenta). C) Representative images of live-cell assay tracking changes in the number of cells over 6 days. Cells were stained with 0.02 μg/mL Hoechst and imaged on days 0 and 6. D-E) Quantification of B and C. F) Rate of multinucleation and G) DNA content analysis after 6 days of treatment. Colors of stacked bar plots indicate ploidy states (2c, 4c, >4c) measured by the integrated intensity of Hoechst 33342. Treatments for (B-G) are as follows: negative control (0.1% DMSO), positive control (1 μM CHIR99021 aka CHIR), and maximum-effect concentrations for top five compounds (3.2 μM C3). H) Functional analogs to C3 and C4 induced cardiomyocyte proliferation. All experiments were performed using lot 3 of CDI iCell Cardiomyocytes. Scale bars are 100 μM. Error bars represent mean±s.e.m. Stats for (D-H) were assessed by one-way ANOVA with Dunnett multiple comparisons test; n=3¬¬−6; *p<0.05, **p<0.01, ***p<0.001.

FIGS. 2A-2F. Functional analogs support putative targets of lead compounds. A) 5-point concentration responses (0.1-10 μM) of functional analogs to each of the top five compounds measuring the change in the number of cells over 6 days of treatment. B) Change in the number of cells, C) rate of multinucleation, D) fraction of diploid nuclei over 6 days of treatment at the maximum-effect concentrations for each compound, E) area under the concentration response curve, and F) concentration at 50% maximum-effect proliferation for each compound. (A-F) Metrics are normalized to the negative control (0.1% DMSO) on each plate. Functional analog compounds (grey) are identified by appending consecutive lower-case letters after lead compound (black) names. All plots are grouped by lead compound. Error bars represent mean±s.e.m. Stats for (B-D) were assessed by one-way ANOVA with Dunnett multiple comparisons test; n=2-24; *p<0.05, **p<0.01, ***p<0.001.

FIGS. 3A-3F. Transcriptomic analysis of proliferation-inducing compounds reveals a common and robust cell cycle response. A) Hierarchical clustering of gene expression profiles and replicates of hiPSC-CMs treated with negative control (0.1% DMSO) and the top five compounds at maximum-effect concentrations (3.2 μM C3). Only genes that are differentially expressed (FDR≤0.05) compared to control in at least one compound are shown. B) Principal component analysis of data shown in (A). C) Distribution of DEGs that are unique (red) to each compound or common among 2-5 compounds. D) Enrichr's Reactome pathway enrichment analysis of the 1764 DEGs common to all five compounds (circles) and 1540 DEGs excluding the Reactome Cell Cycle gene set (squares). Top 15 pathways with FDR≤0.1 are shown. E) Heatmap of log 2 fold change of genes in Reactome's Cell Cycle pathway. Genes that are differentially expressed (FDR≤0.05) in at least one compound are shown. F) ChEA3 transcription factor enrichment analysis of the 1764 common DEGs. Top 15 TFs rank-ordered by FDR with the corresponding mRNA expression profiles are shown. RNAseq was performed with n=3 replicates.

FIGS. 4A-4F. Functional proteomic profiling identifies a mechanistic role for receptor tyrosine kinases in response to pro-proliferative compounds. A) Heatmap of unidirectional differentially expressed RPPA probes common to all 4 lead compounds. Log2 (fold change expression relative to DMSO control) is shown. B) Heatmap of 49 RTK probes measured using R&D's Human RTK array. Proteomic data shown in (A-B) were measured in hiPSC-CMs treated with the top 4 lead compounds (C3) for 12 hrs. C) 48 h EdU assay measuring DNA synthesis rates. D) 6-day live-cell proliferation assay tracking cell counts. E) Rate of multinucleation and F) DNA content after 6 days of treatment. Colors of stacked bar plots indicate DNA content levels (2c, 3c, 4c, >4c) measured by the integrated intensity of DAPI. Stats indicate significance for % 2c nuclei. Treatments for (C-F): negative control (0.1% DMSO), positive control (1 μM CHIR99021), and tyrosine kinase inhibitors targeting ERBB2 (1 μM lapatinib), IGF1R (0.1 μM linsitinib), VEGFR1/2/3 (10 μM axitinib) alone and in combination with 3.2 μM C3. Error bars represent mean±s.e.m. Stats for (C-F) were assessed by one-way ANOVA with Benjamini-Hochberg FDR correction for select comparisons (*=all single treatments were compared to DMSO control; +=all combination treatments were compared to the respective lead compound C3); n=3-9; *p<0.05; **p<0.01; ***p<0.001.

FIGS. 5A-5C. Compound-induced expression of VEGF receptors limits increases in ploidy. A) Ki67 staining measuring cell cycle activity after 6 days of treatment. B) Bivariate analysis of DNA content (x-axis) and Ki67 staining (y-axis) to separate out cell cycle phase and nuclear ploidy levels. DNA content thresholds separating 2c, 3c, and 4c nuclei are shown as blue horizontal lines. Ki67 positive threshold is shown as a red horizontal line. Nuclei with <2c or >4c DNA content are not shown. Replicate with median G0 measurement is shown. C) Quantification of B. Treatments for (A-C): negative control (0.1% DMSO), positive control (1 μM CHIR99021), and small molecule inhibitors targeting ERBB2 (1 μM lapatinib), IGF1R (0.1 μM linsitinib), and VEGFR1/2/3 (10 μM axitinib) alone and in combination with 3.2 μM C3. Error bars represent mean±s.e.m. Stats for (A and C) was assessed by one-way ANOVA with Benjamini-Hochberg FDR correction for select comparisons (*=all single treatments were compared to DMSO control; +=all combination treatments were compared to the respective lead compound C3); n=3-9; *p<0.05; **p<0.01; ***p<0.001.

FIGS. 6A-6D. Network integrating transcriptomic and proteomic data identifies JNK and PI3K pathways mediate compound-induced proliferation of hiPSC-CM. A) Directed molecular network integrating common factors identified in the proteomic and transcriptomic data. Node colors represent the sum of the qualitative significance (up=1, down=−1, not significant=0) across all 4 cpds based on data from the RPPA or RTK array. Grey nodes indicate nodes that were not measured in the RPPA or RTK arrays. B) 48 h EdU assay measuring DNA synthesis rates. C) 6-day live-cell proliferation assay tracking cell counts. D) Ki67 staining measuring cell cycle activity. Treatments for (B-D): neg control (0.1% DMSO), pos control (1 μM CHIR99021), and small molecule inhibitors targeting JNK (10 μM SP600125), MEK (10 μM PD98059), and PI3K (10 μM LY294002) alone and in combination with 3.2 μM C3. Error bars represent mean±s.e.m. Stats for (D-G) were assessed by one-way ANOVA with Benjamini-Hochberg FDR correction for select comparisons (*=all single treatments were compared to DMSO control; +=all combination treatments were compared to the respective lead compound C3); n=3-9; *p<0.05; **p<0.01; ***p<0.001.

FIGS. 7A-7C. Proliferative responses for top five compounds are reproducible across multiple lots. A) 5-point concentration responses (0.1-10 μM) of the top five compounds measuring the change in the number of cells over 6 days of treatment. Data is normalized to the negative control (0.1% DMSO) on each plate. B) Rate of multinucleation and C) DNA content analysis after 6 days of treatment. Colors of stacked bar plots indicate ploidy states (2c, 4c, >4c) measured by the integrated intensity of Hoechst 33342. Treatments for (B-C) are as follows: negative control (0.1% DMSO), positive control (1 μM CHIR99021), and maximum-effect concentrations for top five compounds (3.2 μM C3). All plots are grouped by CDI iCell Cardiomyocyte lot number. Error bars represent mean±s.e.m. Stats for (B-C) were assessed by one-way ANOVA with Dunnett multiple comparisons test; n=3-6; *p<0.05.

FIGS. 8A-8C. Transcriptomic profiling of five lead compounds reveals regulation of distinct pathways. A) Volcano plots of RNASeq gene expression for each lead compound (C3) compared to DMSO control. DEGs are shown in red (FDR≤0.05). B) Enrichr's Reactome pathway and C) ChEA3's transcription factor enrichment analysis of the DEGs shared with at most 2 other compounds. Only the top 5 pathways and transcription factors (rank-ordered by FDR) for each compound are shown.

FIGS. 9A-9C. Nuclei number and circularity decrease after 6 days of VEGFR_i treatment. A) cell number and B) average nuclei circularity of hiPSC-CMs after 2 and 6 days of treatment. Treatments: negative control (0.1% DMSO), positive control (1 μM CHIR99021), and tyrosine kinase inhibitors targeting ERBB2 (1 μM lapatinib), IGF1R (0.1 μM linsitinib), VEGFR1/2/3 (10 μM axitinib) alone and in combination with 3.2 μM C3. All boxplots are grouped by lead compound. Stats were assessed by one-way ANOVA with Dunnett multiple comparisons test; (all single treatments were compared to DMSO control; all combination treatments were compared to the respective lead compound C3 or C4); n=3-9; *p<0.05, **p<0.01, ***p<0.001. C) Images of hiPSC-CMs stained for DAPI and Ki67 where outlines represent automated image segmentation of nuclei. Binucleation is shown with arrows and positive expression of Ki67 is shown as +next to corresponding nucleus. Treatments: negative control (0.1% DMSO), positive control (1 μM CHIR99021), and small molecule inhibitors targeting ERBB2 (1 μM lapatinib), IGF1R (0.1 μM linsitinib), and VEGFR1/2/3 (10 μM axitinib) alone and in combination with 3.2 μM C3. Scale bar=50 μM.

FIGS. 10A-10D. JNK and PI3K mediate compound-induced proliferation without increasing the fraction of multinucleated hiPSC-CMs or cell-cycle arrested polyploid nuclei. A) Multinucleation and B) DNA content analysis after 6 days of treatment. Colors of stacked bar plots indicate DNA content levels (2c, 3c, 4c, >4c) measured by the integrated intensity of DAPI. Stats indicate significance for % 2c nuclei. C) Bivariate analysis of DNA content (x-axis) and Ki67 staining (y-axis) to separate out cell cycle phase and nuclear ploidy levels. DNA content thresholds separating 2c, 3c, and 4c nuclei are shown as blue horizontal lines. Ki67 positive threshold is shown as a red horizontal line. Nuclei with <2c or >4c DNA content are not shown. Replicate with median G0 measurement is shown. D) Quantification of C. Treatments for (A-D): neg control (0.1% DMSO), pos control (1 μM CHIR99021), and small molecule inhibitors targeting JNK (10 μM SP600125), MEK (10 μM PD98059), and PI3K (10 μM LY294002) alone and in combination with 3.2 μM C3. Error bars represent mean±s.e.m. Stats for (A and B) was assessed by one-way ANOVA with Benjamini-Hochberg FDR correction for select comparisons (*=all single treatments were compared to DMSO control; +=all combination treatments were compared to the respective lead compound C3); n=3-9; *p<0.05; **p<0.01; ***p<0.001

FIG. 11. Compound annotations.

### DETAILED DESCRIPTION

The detailed description set forth below, in connection with the appended drawings, is intended as a description of various contemplated methods and usable materials and is not intended to represent the only alternatives by which the subject matter described herein may be practiced. The detailed description includes specific details to provide a thorough understanding of various embodiments of the present disclosure. However, it will be apparent to those skilled in the art that the various features, concepts, and embodiments described herein may be implemented and practiced without these specific details.

In a first aspect, provided herein is a method for inducing proliferation of a cardiomyocyte culture, the method comprising contacting the cardiomyocyte culture with a therapeutic agent selected from:

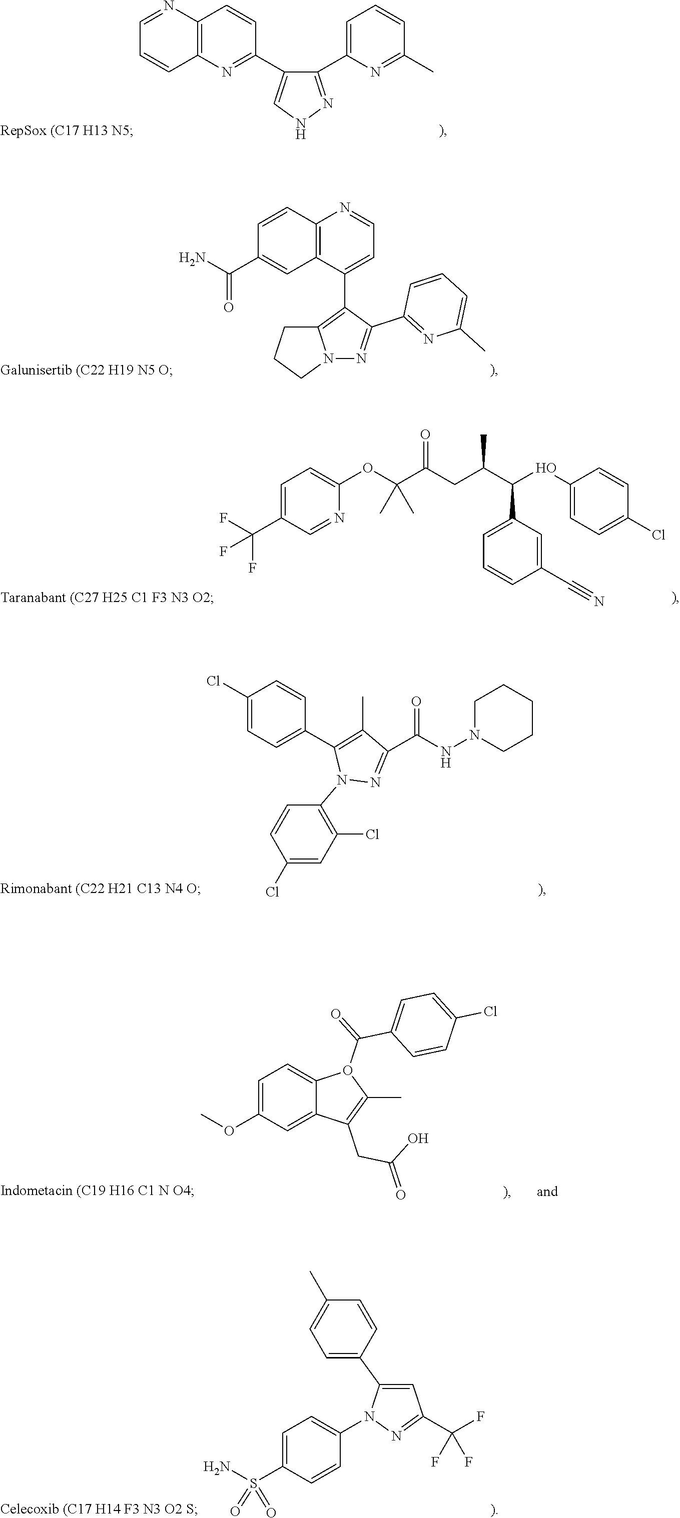

As described in the Examples, human adult hearts contain large populations of polyploid cardiomyocytes, which have lower proliferative capacities and limit cardiac regeneration. The inventors have discovered that RepSox, also called C3 herein, and analogs thereof, and other similar compounds are able to stimulate proliferation in polyploid cardiomyocytes. As used herein, the term “therapeutic agent” refers to a chemical compound that stimulates proliferation of cardiomyocytes.

The term “culture” refers to a population of cells grown under controlled conditions in vitro. A cardiomyocyte culture may comprise a cardiomyocyte cell line, e.g. the iCell cardiomyocyte cell line. A cardiomyocyte culture may comprise a primary cardiomyocyte cell. The cardiomyocyte culture may comprise binucleated or polyploid cardiomyocytes. The cardiomyocyte culture may comprise human or non-human mammalian cardiomyocytes. In exemplary embodiments, contacting the cell culture with the therapeutic agent does not increase the amount of binucleated or polyploid cardiomyocytes.

“Contacting” a cell or a cardiomyocyte culture refers to adding the therapeutic agent to a medium or buffer comprising the cell culture. The therapeutic agent may be contacted to the culture at a concentration of between about 0.1 and about 10 μM, or any concentration or range in between. When the therapeutic agent is RepSox, the concentration may be about 3 μM. In exemplary embodiments, the RepSox is provided at about 3.2 μM.

In a second aspect, provided herein is a method for inducing expression of at least one of VEGFR2 and ErbB2 expression in a cell, the method comprising contacting the cell with RepSox. The RepSox, may be provided at a concentration of between about 0.1 and about 10 μM, or any concentration or range in between. The concentration may be about 3 μM. The cell may be a cardiomyocyte. To “induce expression” is to increase the amount of RNA and/or protein.

In a third aspect, provided herein is a method for treating heart damage in a subject in need thereof, the method comprising administering to the subject an effective amount of a therapeutic agent selected from RepSox, Galunisertib, Taranabant, Rimonabant, Indometacin, or Celecoxib, or a pharmaceutically acceptable salt thereof.

As used herein, the term “administering”, refers to dispensing, delivering, or applying the therapeutic agent, to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The terms “treating” and “to treat” includes the reducing, repressing, delaying, repairing, mitigating, or preventing a disease, disorder, ailment, or condition.

The heart damage may be caused by side effects of a treatment, drug, pathogen, or other external factor to which a subject is exposed. As an example, certain chemotherapy agents for treatment of cancer are known to have cardiotoxic effects. Thus, prior to, during, and/or after chemotherapy, the subject may also be administered a therapeutic agent described herein to promote cardiomyocyte activity to repair damage, to prepare for or prevent heart damage, or to mitigate ongoing damage caused by the chemotherapy. Other more targeted cancer therapies, such as receptor tyrosine kinase (RTK) inhibitory therapy may also cause heart failure, myocarditis, etc., and subjects being treated from such therapies may also be administered therapeutic agents as described herein. Similarly, certain patients being administered other drugs for other conditions known to increase risk of heart failure, stroke, heart attack, aortic aneurysm or the like (e.g., certain categories of anti-inflammatories, stimulants, antibiotics, etc.) may also be treated with the therapeutic agents described herein.

The disclosure also contemplates methods for addressing damage to cardiac tissue from non-treatment factors, such as recreational drug use. In one aspect, a subject undergoing recovery may be treated for withdrawal as well as a therapeutic agent described herein to promote cardiomyocyte proliferation.

Therefore, in some embodiments, the method may further comprise administering an additional therapy, e.g. chemotherapy, RTK inhibitor therapy, heart disease, etc. The therapeutic agent described herein may be administered before, during, or after the additional therapy.

In a fourth aspect, a subject with various risk factors for heart disease, heart attack, etc. that are not necessarily related to heart tissue damage (e.g., scar tissue) may be administered a therapeutic agent described herein to promote cardiac regeneration and health as a means of surviving cardiac events (e.g., survival of sudden cardiac arrest, arrhythmias, etc.).

The therapeutic agent may be prepared as a formulation or pharmaceutical composition. Inert ingredients and manner of formulation of the pharmaceutical compositions are conventional. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). The pharmaceutical compositions may be designed or intended for oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. In embodiments, aqueous and non-aqueous liquid or cream formulations are delivered by a parenteral, oral or topical route. In embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. The pharmaceutical compositions may be lyophilized. The pharmaceutical compositions may contain auxiliary substances such as wetting or emulsifying agents, pH buffering agents, gelling or viscosity enhancing additives, preservatives, flavoring agents, colors, and the like, depending upon the route of administration and the preparation desired. For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of injectability. The composition should be stable under the conditions of manufacture and storage, and must be shielded from contamination by microorganisms such as bacteria and fungi.

The composition may further comprise a pharmaceutically acceptable carrier. The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The terms “small molecule drug” and “chemical compound” as used herein refer to molecules that are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets.

Pharmaceutically acceptable carriers are well known to those skilled in the art and include, but are not limited to, 0.01 to 0.1 M, or 0.05M phosphate buffer, or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Commonly used buffers are phosphates, carboxylates, and bicarbonates. Buffering agents included, but are not limited to, sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, or about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, or within 5% of a given value or range of values. Alternatively, and particularly in biological systems, the terms “about” and “approximately” may mean values that are within an order of magnitude, or within 5-fold and or within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the chemical compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The chemical compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The preferred route may vary with, for example, the subject's pathological condition or age or the subject's response to therapy or that is appropriate to the circumstances. The formulations can also be administered by two or more routes, where the delivery methods are essentially simultaneous, or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. In some embodiments, the optimum effective amount can be readily determined by one skilled in the art using routine experimentation.

In a fifth aspect, provided herein is a method for treating a subject in need thereof, the method comprising administering to the subject an effective amount of a therapeutic agent selected from RepSox, Galunisertib, Taranabant, Rimonabant, Indometacin, or Celecoxib, or a pharmaceutically acceptable salt thereof, wherein the subject is genetically predisposed to a heart defect. Genetic heart defects are caused by mutations in one or more genes. Therefore, the therapeutic agent may be administered in combination with a gene therapy. In another example, a therapeutic agent described herein may be administered to a subject in utero or otherwise in early development when a cardiac defect is detected.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The cited references are incorporated by reference herein in their entireties. In the event that there is an inconsistency between a definition of a term in the specification as compared to a definition of the term in a cited reference, the term should be interpreted based on the definition in the specification.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### INTRODUCTION

Technological advances in high-content imaging and automated image analysis have enabled rapid and efficient screening of therapeutic agents that enhance cardiomyocyte proliferation. Previous phenotypic screens of chemical compounds and microRNAs have identified numerous agents that stimulate cell cycle reentry and progression in murine [1, 2] and human [3-5] cardiomyocytes, with many focusing on classical endpoint cell cycle markers (e.g. BrdU/EdU or Ki67). Postnatal mammalian cardiomyocytes can progress through unconventional cell cycles, such as endoreplication cycles, resulting in binucleated or polyploid phenotypes. Thus, assays using early cell cycle markers conflate cell cycle activity and authentic cardiomyocyte proliferation. Mature adult rodent [6, 7] and human [8] hearts contain significant populations of binucleated or polyploid cardiomyocytes, respectively. Studies have reported binucleated and polyploid cardiomyocytes have lower proliferative capacities and limit cardiac regeneration in vivo compared to mononucleated diploid cardiomyocytes [6, 9, 10]. However, the biological significance of binucleation or polyploidization in the heart has yet to be determined. To address these limitations, we previously designed a high-content live-cell proliferation assay that discriminates between multiple cell cycle variants that generate new mononucleated diploid, binucleated, and polyploid human induced pluripotent stem cell-derived cardiomyocytes (hiPSC-CMs) [11].

While numerous targets, compounds, and microRNAs have been identified through phenotypic screens, the molecular mechanisms underlying the proliferative responses of such agents are often unknown [1, 2, 5, 12, 13]. A high-content phenotypic screen using a 3D human cardiac organoid system identified compounds that induced hiPSC-CM cell cycle reentry through mitosis without inducing binucleation or negatively impacting contractile properties of the cardiac organoids [5]. Additional transcriptomic and proteomic analysis of four pro-proliferative compounds hitting distinct pathways revealed the mevalonate pathway as a core mediator of cardiomyocyte cell cycle progression [5]. Another group used high-content screens, RNA sequencing, and proteomic arrays to reveal cardiotoxic VEGFR2/PDGFR tyrosine kinase inhibitors triggered the compensatory activation of insulin/IGF1 signaling and the upregulation of VEGF receptor genes, that ultimately could be harnessed to promote hiPSC-CM survival [14]. These studies highlight unbiased molecular and phenotypic screening approaches that translate targets to mechanisms.

We previously performed a series of fixed and live-cell phenotypic screens that identified compounds inducing DNA synthesis and proliferation without enhancing binucleation or polyploidization in hiPSC-CMs [11]. In this study, we combine high-content phenotypic profiling and high-throughput multi-omic strategies to discover key mechanisms by which a diverse set of compounds stimulate proliferation in hiPSC-CMs. We selected five compounds, that were highly active in our phenotypic screens, with diverse putative targets including WNT, BRAF, ALK5, CB1R, and COX1/2. Putative targets represent the known compound targets that might mediate hiPSC-CM proliferation. Transcriptomic and proteomic analyses of these compounds identified a common set of differentially regulated transcription factors, proteins, and pathways including multiple receptor typrosine kinases (RTKs). Network analysis integrating the common multi-omic signatures suggested multiple compound signals converged on canonical RTK pathway mediators INK, MEK, and PI3K to promote hiPSC-CM proliferation. Additional perturbation experiments revealed key mechanisms regulating cardiomyocyte reentry, cell division, binucleation, and polyploidization.

### Results

Diverse lead compounds induce proliferation without multinucleation or polyploidy in hiPSC-CMs.

To investigate core mechanisms driving proliferation in human iPSC-derived cardiomyocytes (hiPSC-CMs), we selected five highly active compounds with diverse putative targets from a previous study that screened for DNA synthesis, proliferation, and endoreplication [13]. Target annotation data indicated the lead compounds targeted WNT, BRAF, ALK5 (RepSox), CB1R (AZD2691), and COX-1/5-LO. One illustrated compound is RepSox (AZ #446859-33-2), referred to herein as compound C3. C3 has the chemical structure

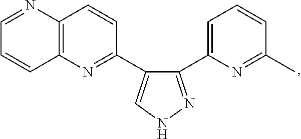 [SMILES: Cc1cccc(-c2n[nH]cc2-c2ccc3ncccc3n2)n1]

and has the putative target ALK5.

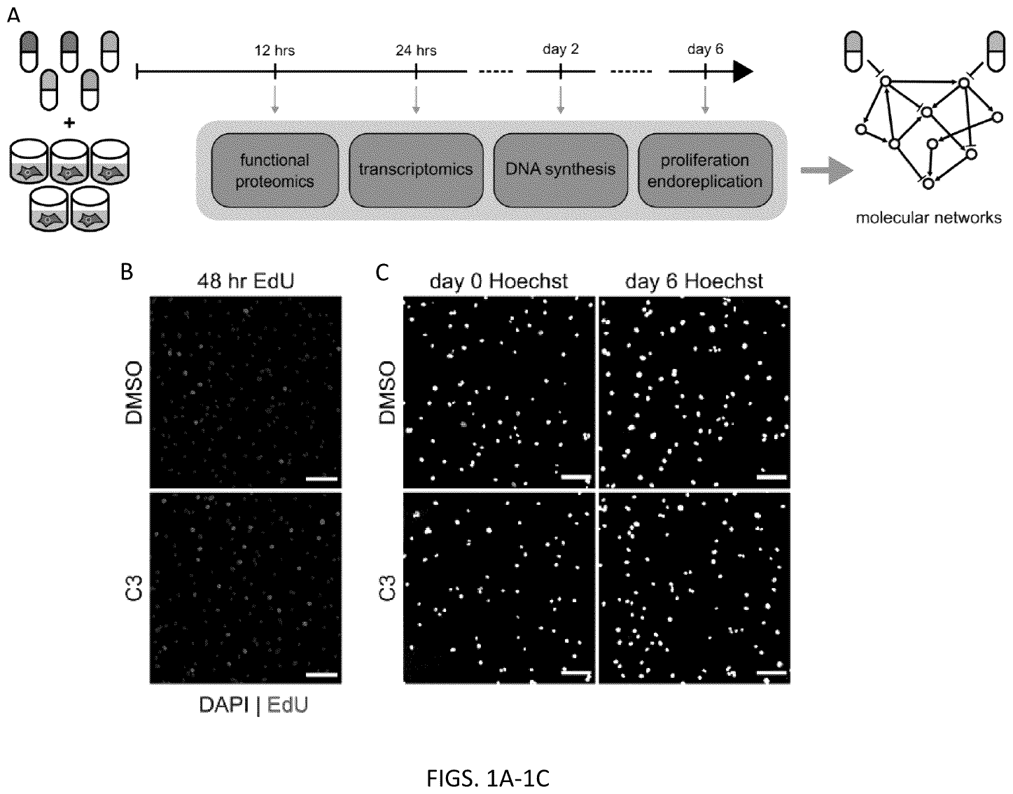

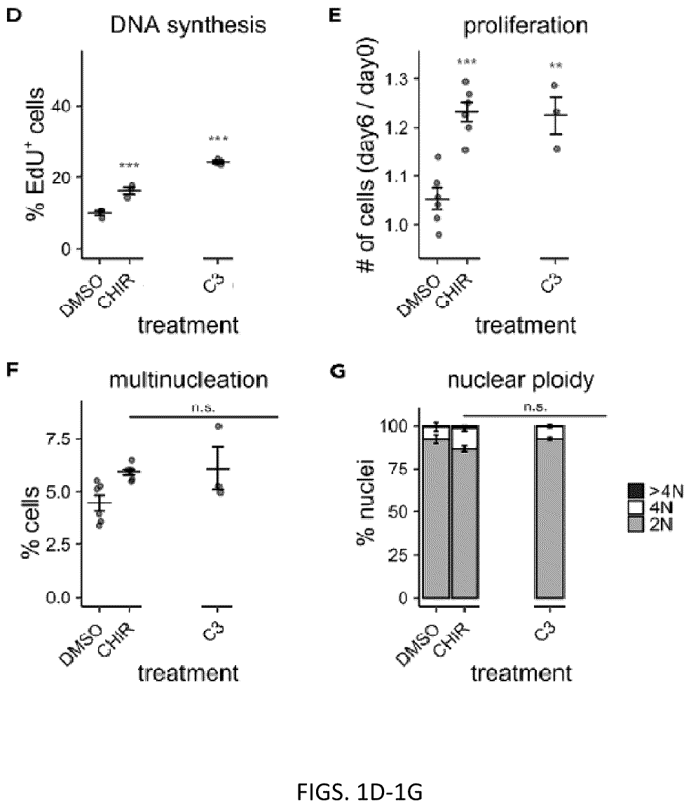

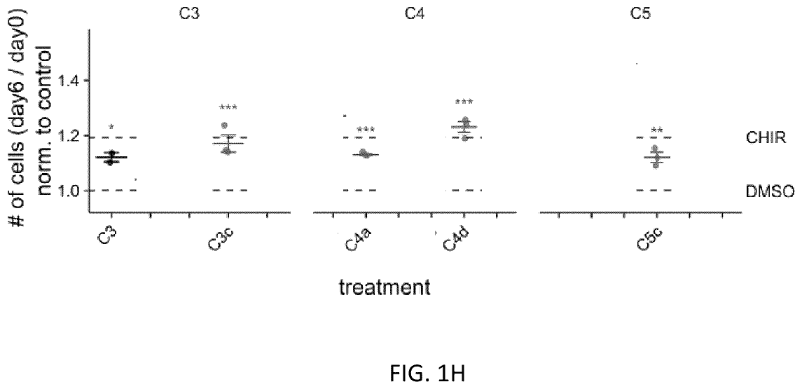

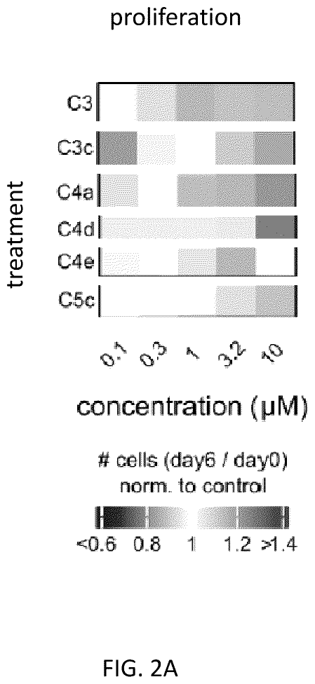

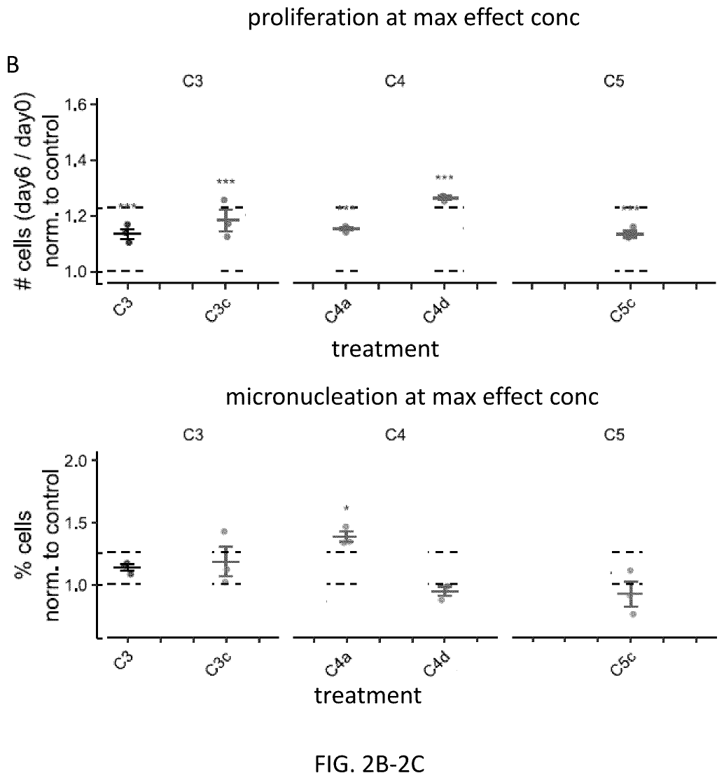

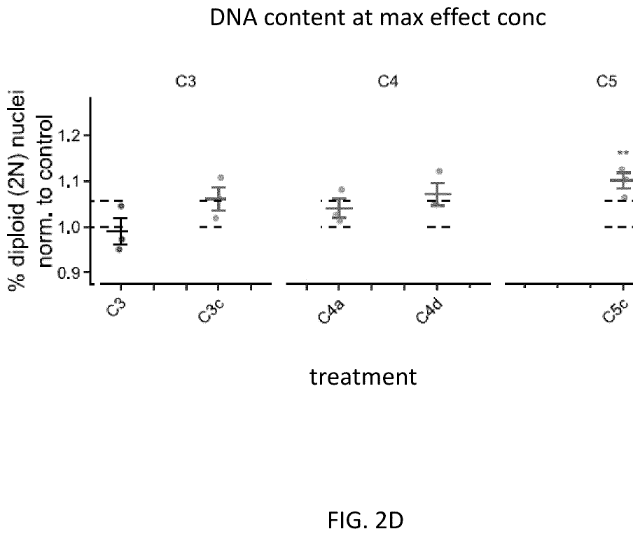

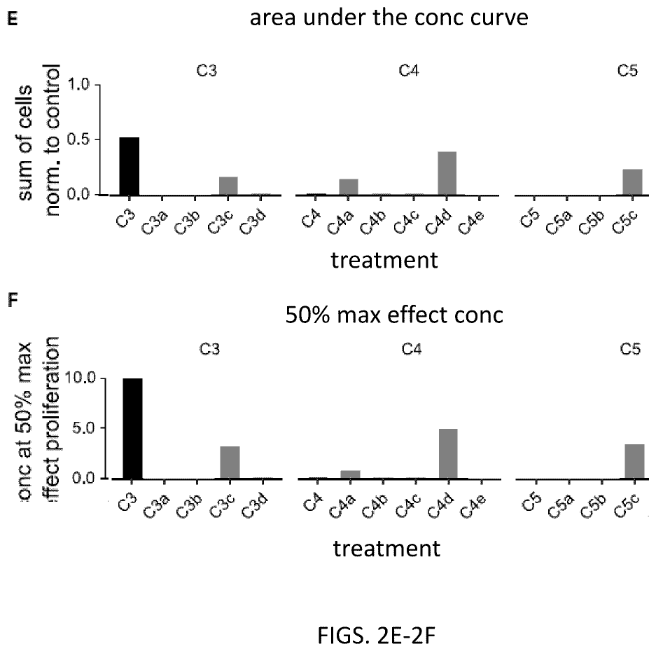

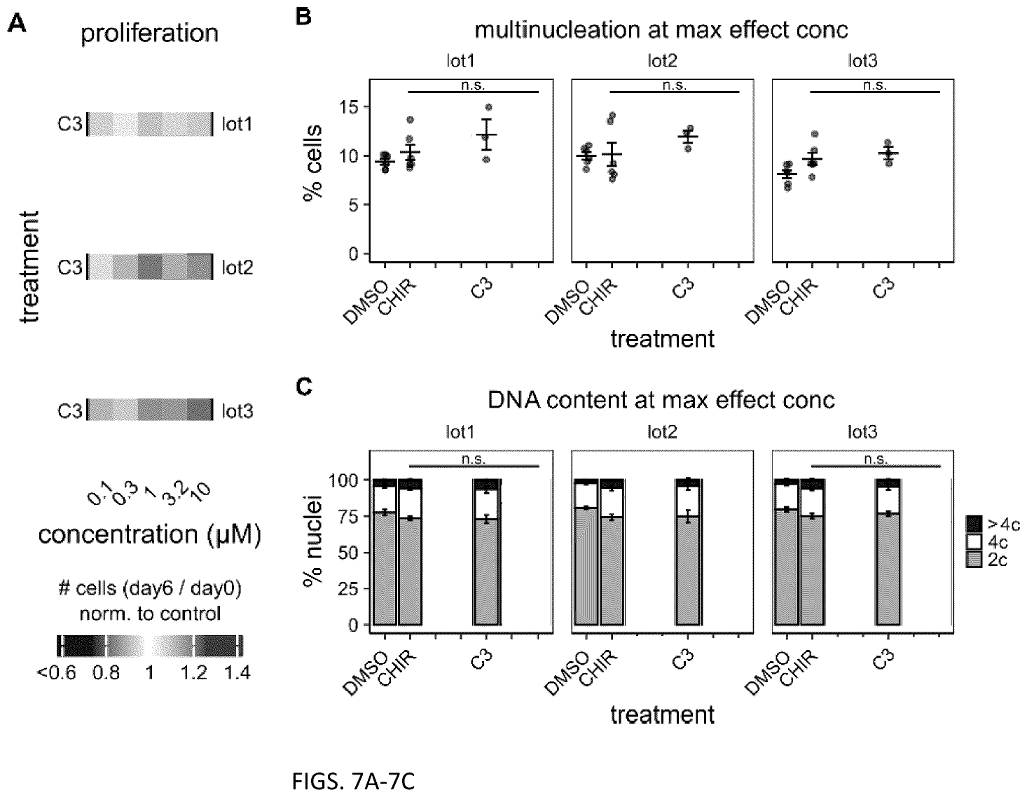

Using the same live-cell proliferation assay we developed in the previous study [13], we confirmed the 5-point concentration-response profiles (0.1-10.0 μM) in three independent manufacturing lots of iCell Cardiomyocytes (FIG. 7). Briefly, hiPSC-CMs were stained with a low, non-toxic concentration of Hoechst 33342 and the same imaging fields of view were collected at both initial (day 0) and final (day 6) timepoints. We quantified the change in the number of cells, binucleation events, and DNA content distribution using the image segmentation and analysis pipeline described in the previous study [13]. Consistent with the previous screens, all five compounds induced both DNA synthesis (FIGS. 1B and 1D) and proliferation (FIGS. 1C and 1E) without increasing the fraction of multinucleated cells (FIGS. 1F and 7) or polyploid nuclei (FIGS. 1G and 2C) at their respective maximal-effective (Emax) concentrations. Functional analogs for compound C3, e.g. C3a-C3d, exhibited even greater potency or efficacy than their corresponding lead compound (FIGS. 2E and 2F). Nonetheless, these diverse compounds stimulated proliferation of hiPSC-CMs.

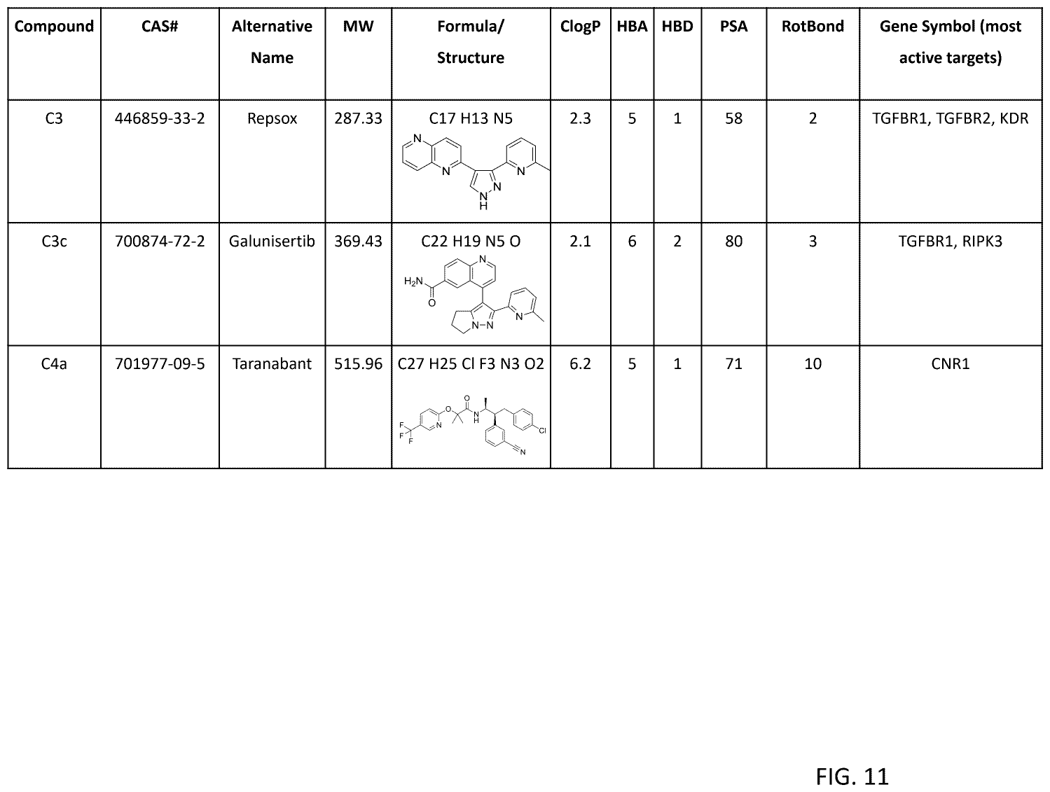

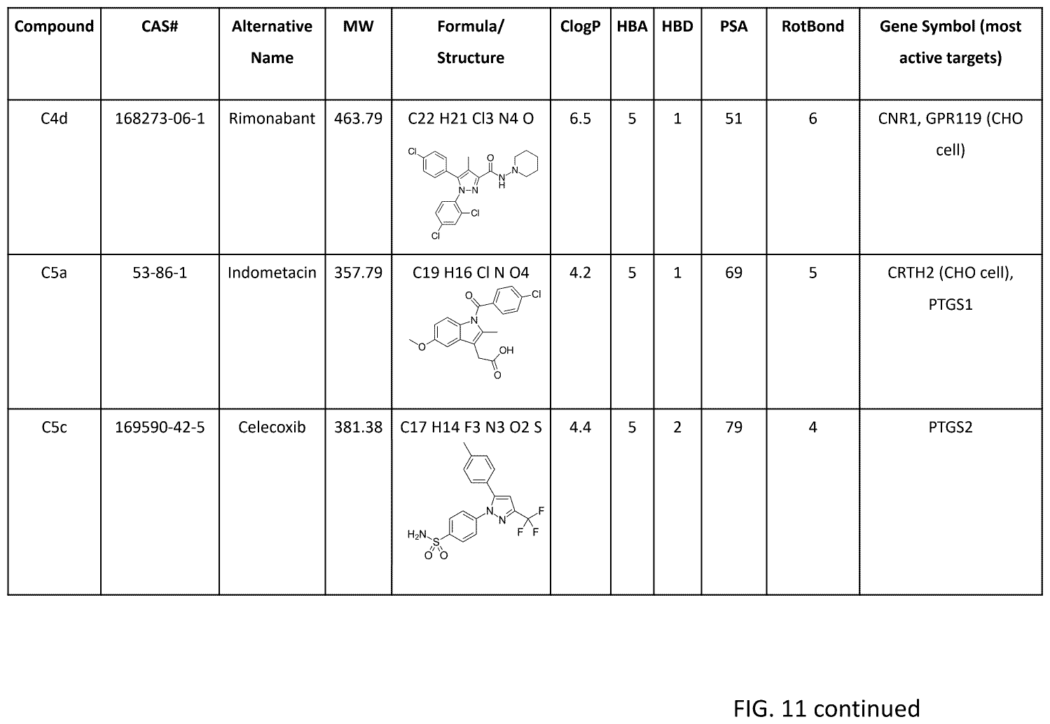

To build our confidence in the putative targets, we tested three to five additional internal and commercially available functional analogs (FIG. 11) that have the same putative targets as each of the 5 lead compounds across five concentrations (FIG. 2A).

Using the live-cell proliferation assay, we confirmed more than two functional analogs for C3, which significantly increased proliferation over 6 days at Emax concentrations (FIG. 2B), without increasing the rate of multinucleation (FIG. 2C) or polyploidy (FIG. 2D). Together, the above results demonstrate that C3 induces proliferation without additional endoreplication cycles in hiPSC-CMs. Additionally, based on their annotations and results with functional analogs, these results suggest that ALK5, CB1R, and COX-1/5-LO are regulators of hiPSC-CM proliferation.

### Transcriptomic Analysis Exposes how Lead Compounds Support a Robust Cell-Cycle Response

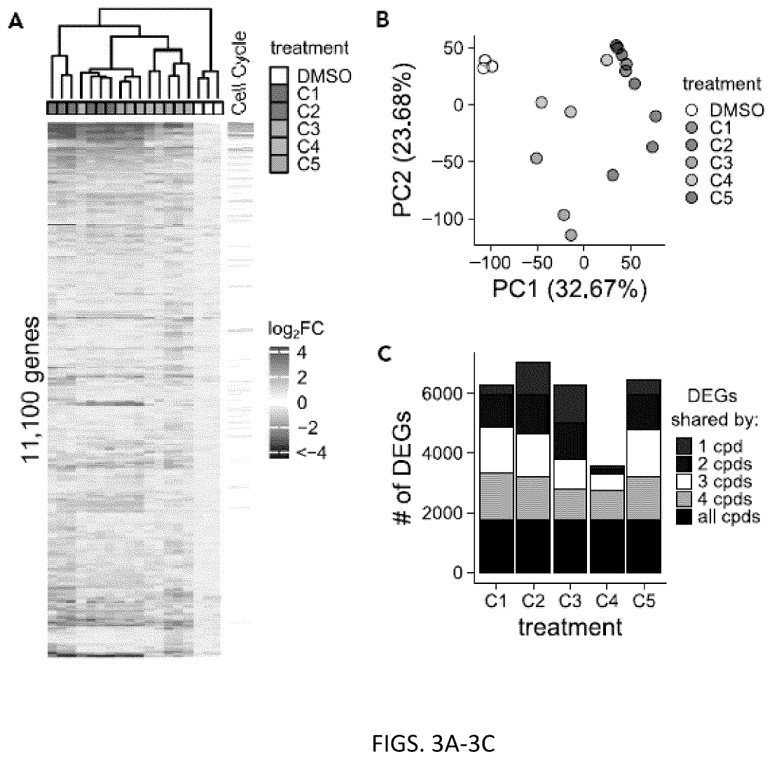

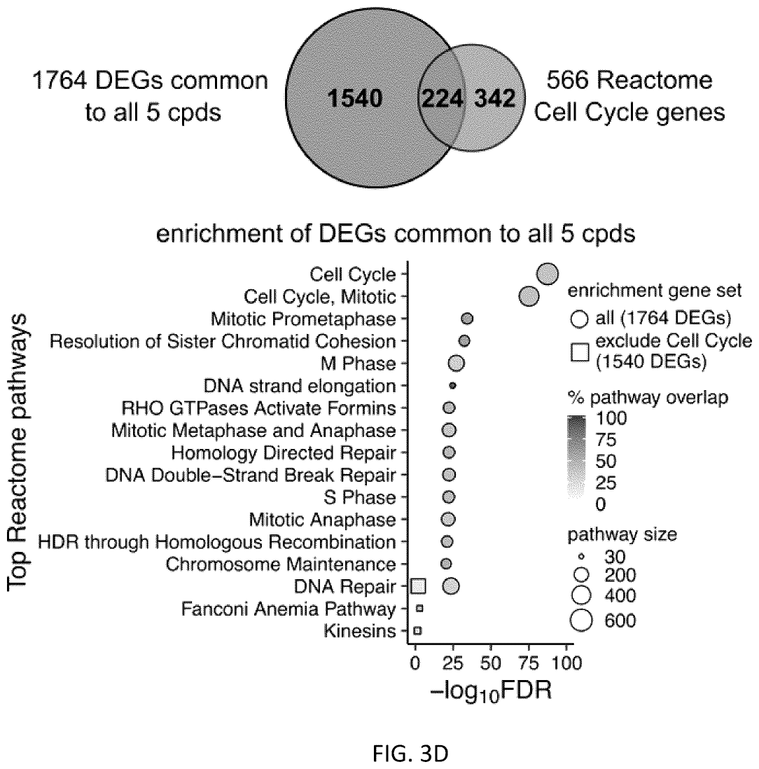

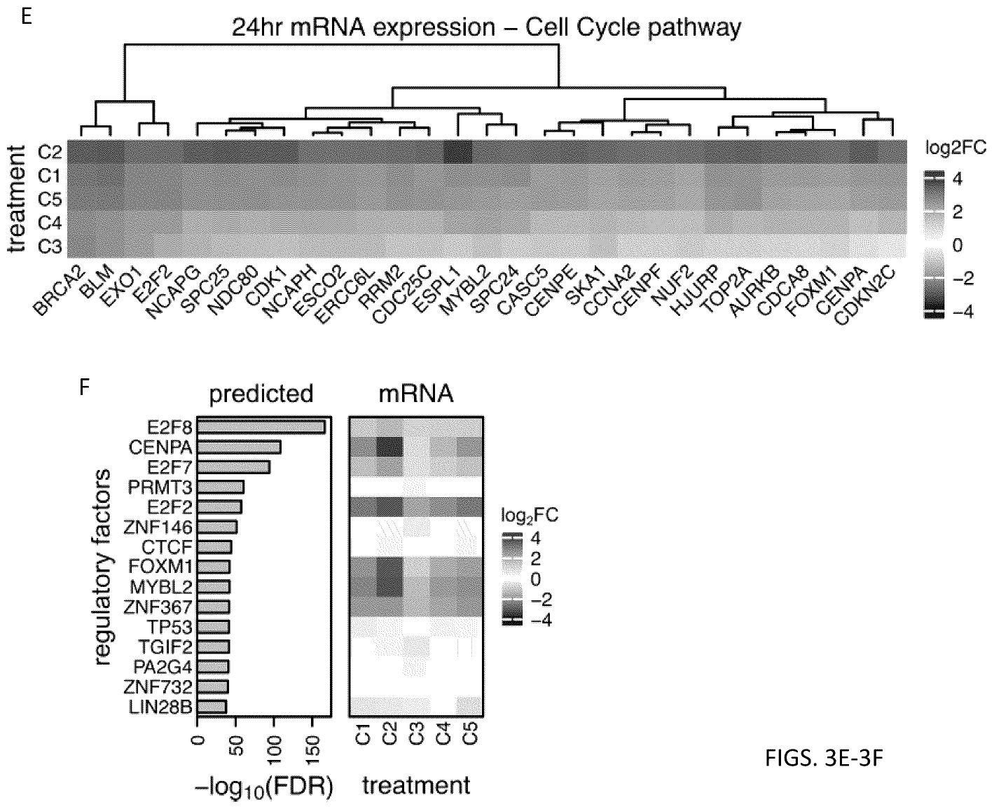

To elucidate molecular mechanisms driving the proliferative responses observed in the phenotypic assays, we performed bulk RNA sequencing on hiPSC-CMs treated with each of the five lead compounds. We sequenced the mRNA isolated from cells after 24 h of treatment at Emax concentrations. Over 11,000 differentially expressed genes (DEGs) were detected in at least one treatment compared to the DMSO negative control. Hierarchical clustering (FIG. 3A) and principal component analysis (FIG. 3B) of the transcriptome profiles demonstrated high correlation within treatment groups, distinct from the negative control group. Differential expression analysis identified between ˜3,500 to 7,000 DEGs for each compound, with 1,764 DEGs common to all five lead compounds (FIG. 3C). We focused our attention on this group of common DEGs.

We next performed pathway enrichment analysis on the 1,764 common DEG set using Enrichr's bioinformatics tool and the Reactome database [17, 18] to identify core pathways involved in regulating proliferation. In this common DEG set, 203 of 1,530 Reactome pathway modules were significantly enriched (adjusted p≤0.05). Consistent with the robust proliferation observed in the phenotypic experiments, Reactome's Cell Cycle module was the most significantly enriched (adjusted p 3.3×10-88) by the common DEG set. In fact, the top 15 significantly enriched pathway modules by the common DEG set were related to cell cycle or DNA repair (FIG. 3D). Cell cycle was also the most significantly enriched pathway using the KEGG 2019 Human library (adjusted p 2.6×10-24), with 224 Cell Cycle genes expressed concordantly (FIG. 3E). While the Reactome Cell Cycle genes accounted for only 12.7% of the common DEGs, excluding the Cell Cycle genes from the common DEG set reduced the total number of significantly enriched pathways down to three—DNA Repair, Fanconi Anemia Pathway, and Kinesins (FIG. 3D).

To identify factors that may be driving these transcriptional responses, we next performed transcription factor (TF) enrichment analysis on the common DEG set using ChEA3's algorithm [19]. The top 15 predicted transcription factors included known cardiomyocyte cell-cycle regulators (e.g. E2F2 [20, 21], FOXM1 [22-24], and MYBL2 [25]) as well as transcriptional regulators not previously reported in the context of hiPSC-CM proliferation including E2F8 and CENPA (FIG. 3F).

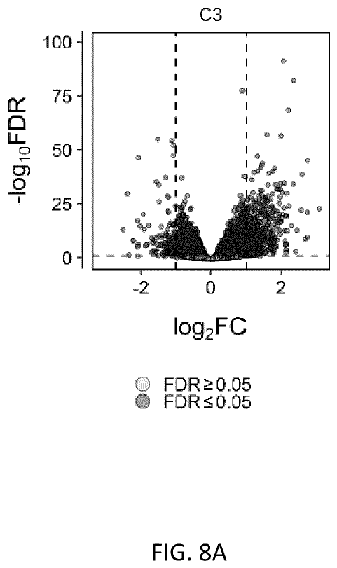

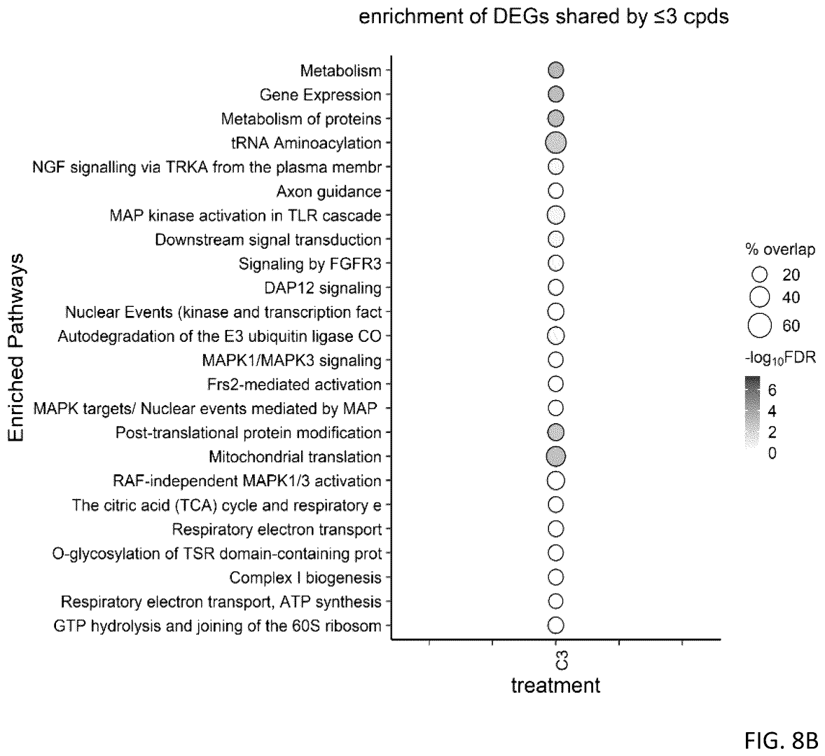

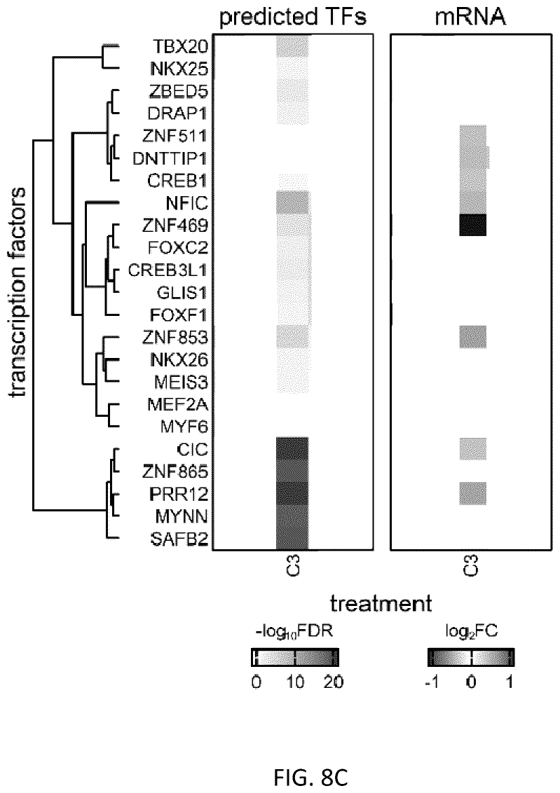

While DEGs common to all the lead compounds regulated cell cycle and DNA repair pathways, the compounds also elicited distinct transcriptional responses related to metabolism, FGF, and MAPK signaling (FIG. 8B). Additionally, cell cycle pathways were noticeably absent from enrichment analysis performed with DEGs that are common to no more than 3 compounds. This suggests the commonalities among all lead compounds are tightly linked to cell cycle regulation.

Functional proteomic profiling reveals mechanistic role for receptor tyrosine kinases in regulating proliferation and endoreplication in response to compounds.

We next analyzed the proteomic responses of hiPSC-CMs to the lead compounds to identify upstream proteomic signatures that may mechanistically link to downstream transcription.

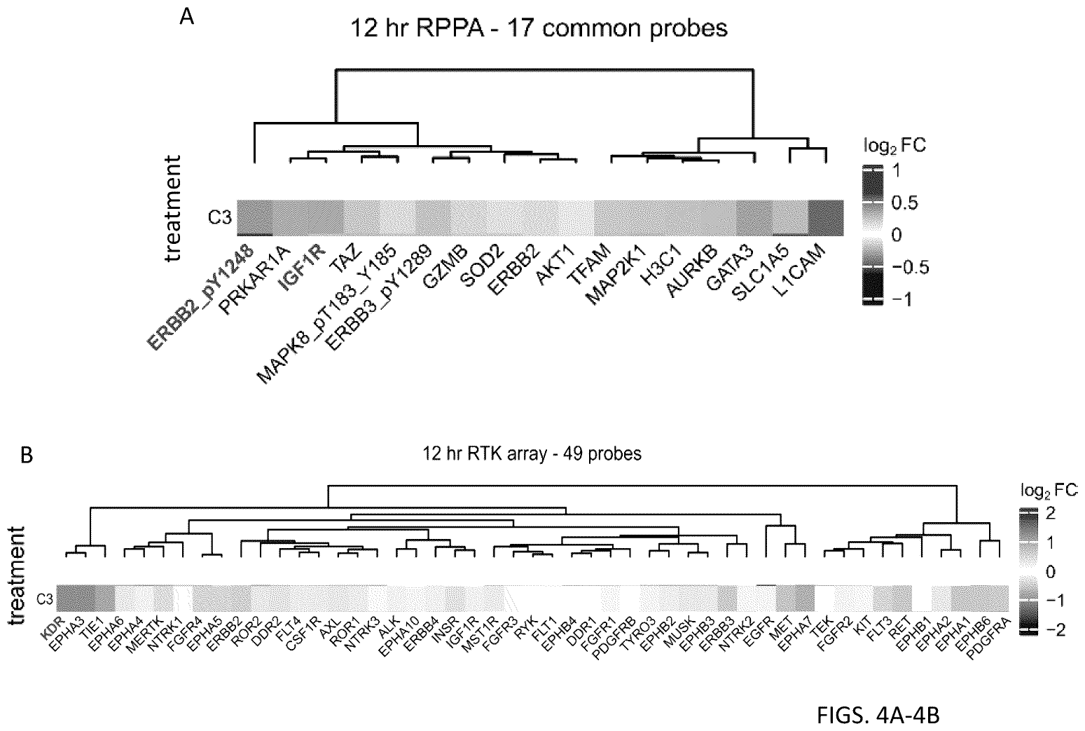

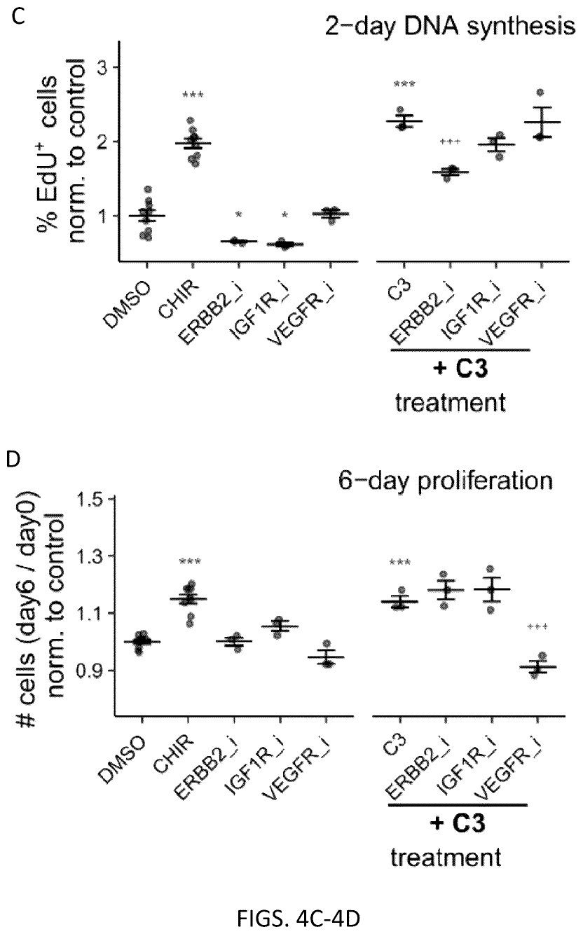

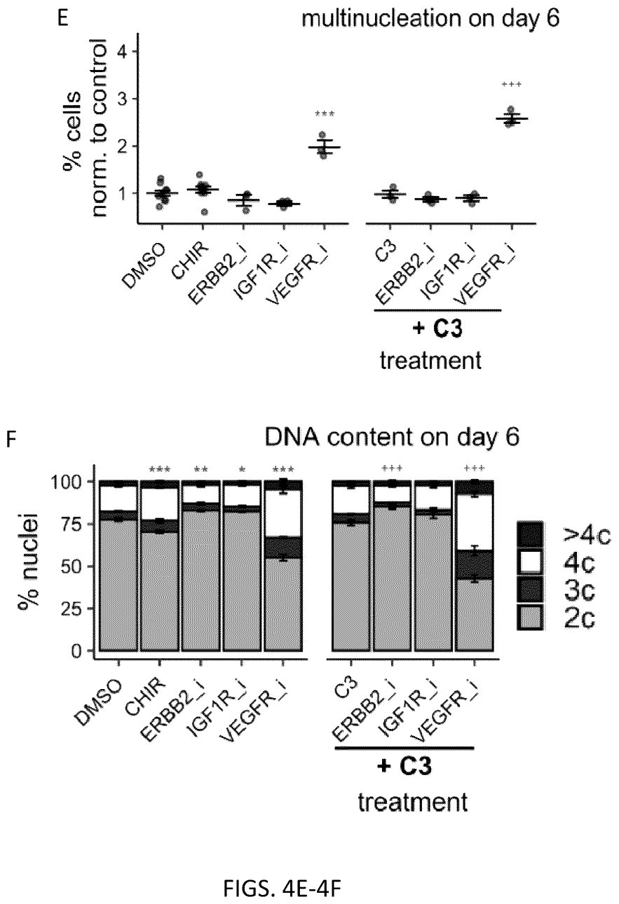

We used reverse phase protein arrays (RPPA) to measure the expression levels and phosphorylation status of 305 proteins in hiPSC-CMs treated for 12 hrs with C3 at Emax concentrations. We found 109 significantly differentially expressed (FDR≤0.05) probes in response to at least one compound (FIG. 4A). Importantly, a common set of 17 probes exhibited concordant responses across all four compounds (FIG. 4B). These probes include upregulation of receptor tyrosine kinases (RTKs) including ErbB2 phosphorylation at Y1248 and total IGF1R expression. To further investigate the role of RTKs in compound-induced proliferation, we profiled the activity of 49 RTKs using a specialized array with the same protein lysates used for the RPPA. Consistent with the RPPA results, the RTK array confirmed the activation of ErbB2 and IGF1R in response to multiple compounds. VEGFR2 (KDR) was also consistently activated across all four compounds compared to the DMSO negative control (FIG. 4C). Thus, despite having distinct molecular targets, these compounds induced common activation of RTKs including ErbB2, IGF1R, and VEGFR2.

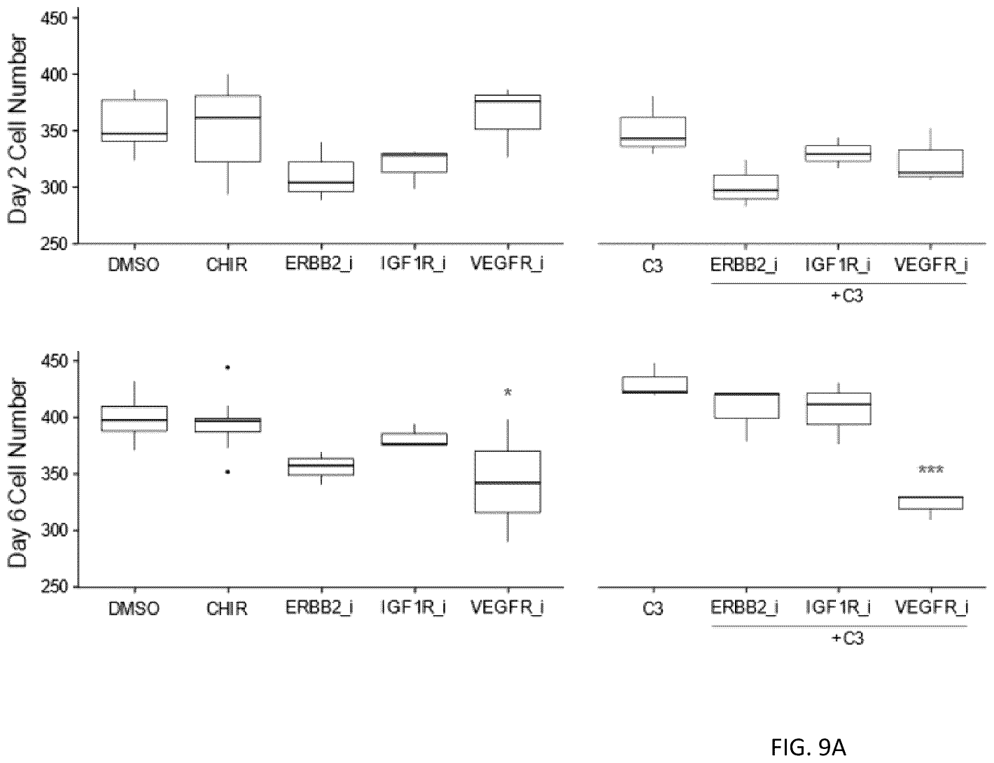

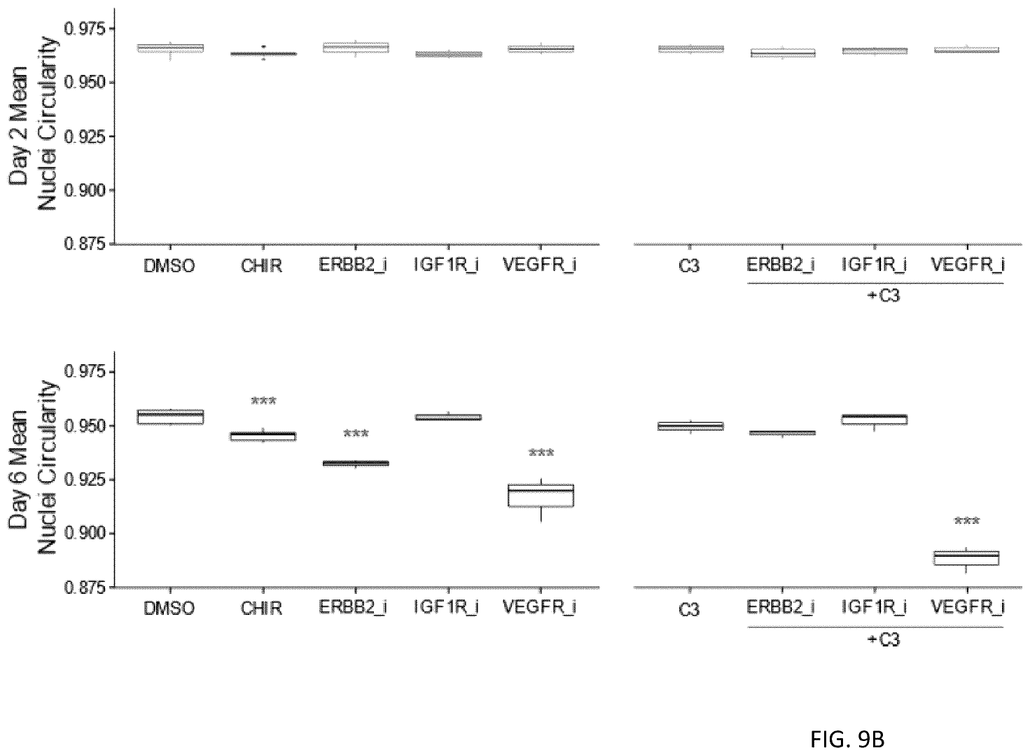

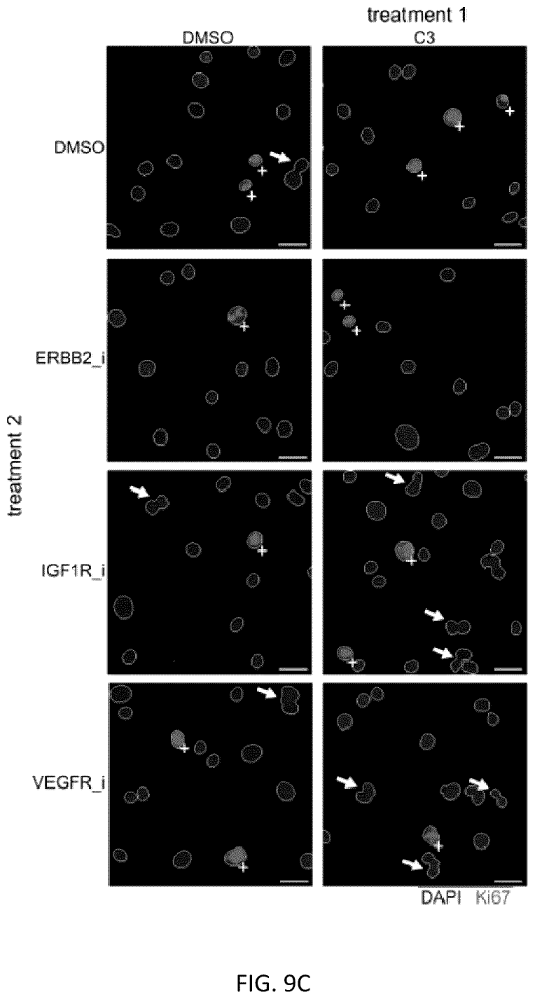

All three of these RTKs have been shown to be highly expressed in hiPSC-CMs [14], and ErbB2 [26-28] and IGF1R [29, 30] have been previously implicated in promoting cardiomyocyte proliferation in other contexts. Furthermore, a phenotypic screen of growth factors revealed that VEGF ligands enhanced proliferation of human iPSC-derived cardiac progenitor cells [31]. However, studies investigating the role of VEGF signaling in cardiomyocyte proliferation have been inconclusive and contradictory [32-34]. Thus, we decided to investigate the mechanistic role of RTK activation in mediating compound-induced proliferation. Tyrosine kinase inhibitors lapatinib, linsinitib, and axitinib were selected to inhibit the activation of ErbB2/EGFR, IGF1R, and VEGF receptors, respectively. We used the 2-day EdU and 6-day live-cell proliferation assays to measure the effects on cell cycle progression in hiPSC-CMs. We found that inhibiting ErbB2 with 1 μM lapatinib attenuated C3 induced DNA synthesis at 48 hrs (FIG. 4D), but had little to no effect on proliferation by day 6 (FIG. 4E). Similarly, inhibiting IGF1R with 0.1 μM linsitinib had a modest effect in reducing DNA synthesis (FIG. 4D) and no effect on late-stage proliferation metrics (FIG. 4E). Conversely, the VEGF receptor inhibitor (10 μM axitinib) alone or in combination with C3 had little to no effect on early DNA synthesis (FIG. 4D), but abrogated C3 induced increase in cell numbers (FIG. 4E). Surprisingly, inhibition of VEGF receptor resulted in over 2-fold increases in multinucleation (FIG. 4F) and substantial decreases in the population of 2c nuclei (FIG. 4G) compared to either the negative control or compound treatments alone. While VEGF receptor inhibition reduced cell numbers at times associated with cell cycle entry, by day 6, VEGFR inhibition in C3-treated hiPSC-CMs decreased nuclei number and circularity (FIG. 9). Thus, the KDR expression induced by C3 may aid later proliferation but also induce cell survival pathways that limit stress-induced multinucleation and polyploidy.

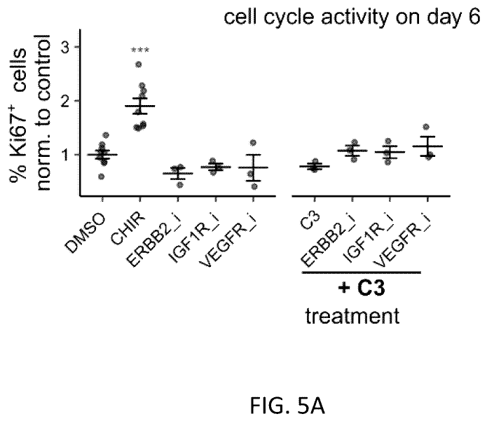

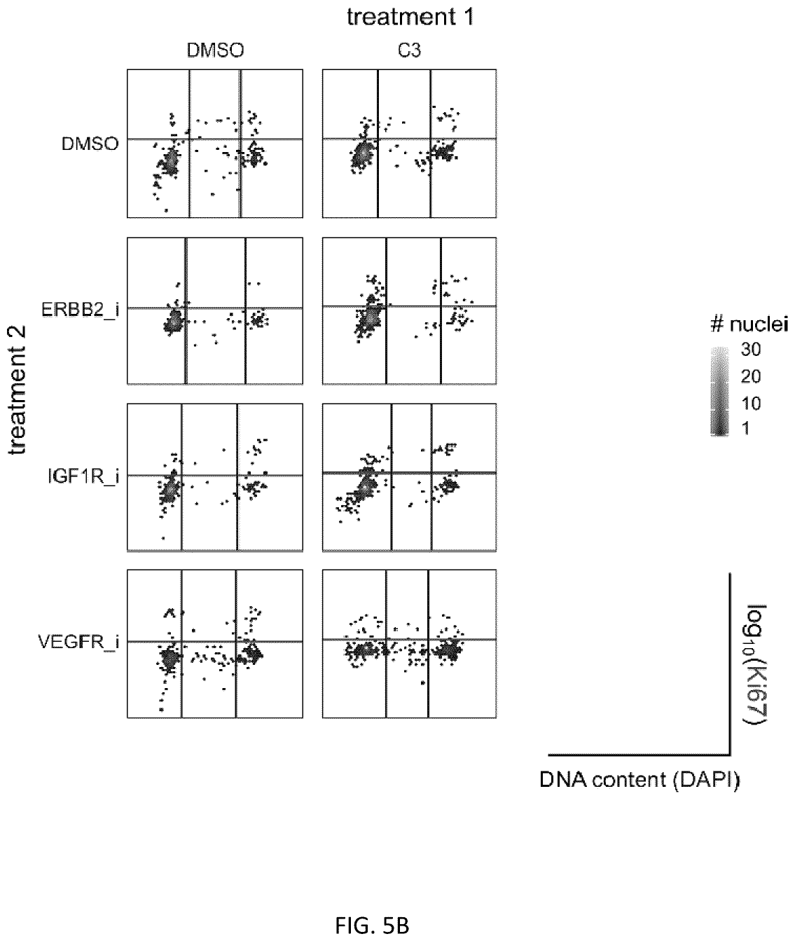

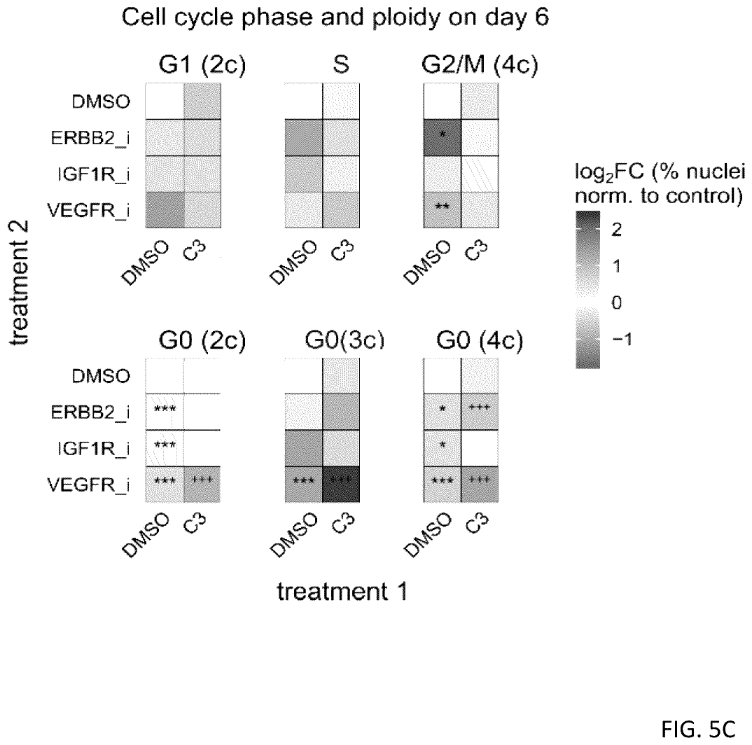

Bivariate analysis of DNA content and Ki67 revealed this decrease in the diploid nuclei population was due to the decrease in the fraction of G0 diploid nuclei and corresponding increases in G0 intermediate DNA content and tetraploid populations (FIGS. 5B, 5C, and 9C). Inhibition of these RTKs had no effect on overall cell cycle activity by day 6 (FIG. 5A). Collectively, these results indicate upregulation of VEGF receptor plays an important role in preventing defects in cytokinesis, karyokinesis, and chromosome segregation in proliferating hiPSC-CMs. These results also suggest that C3 mediates DNA synthesis and bona-fide proliferation via the activation of ErbB2 and VEGF receptors, respectively.

Network Integration of Multi-Omic Data Identifies Pathways that Converge on JNK and PI3K

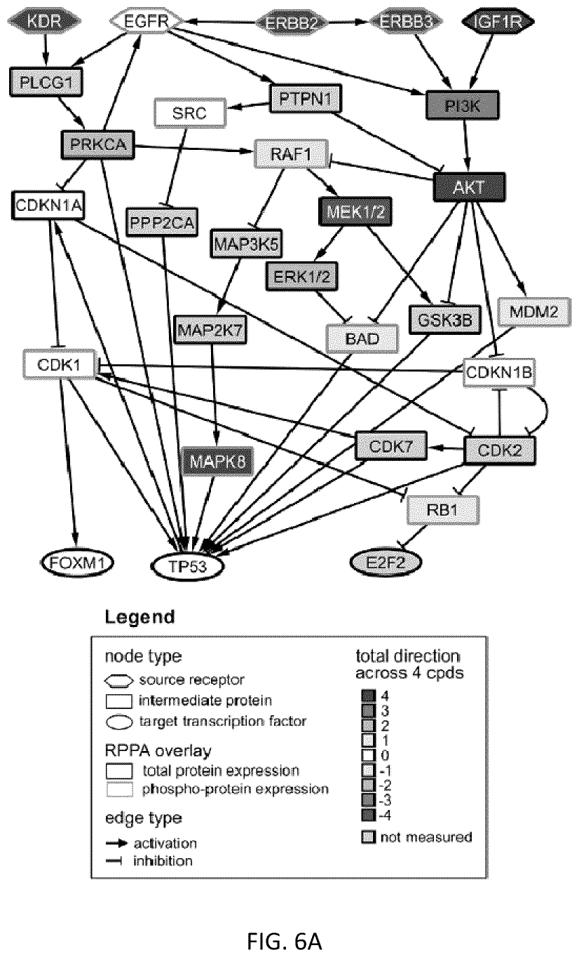

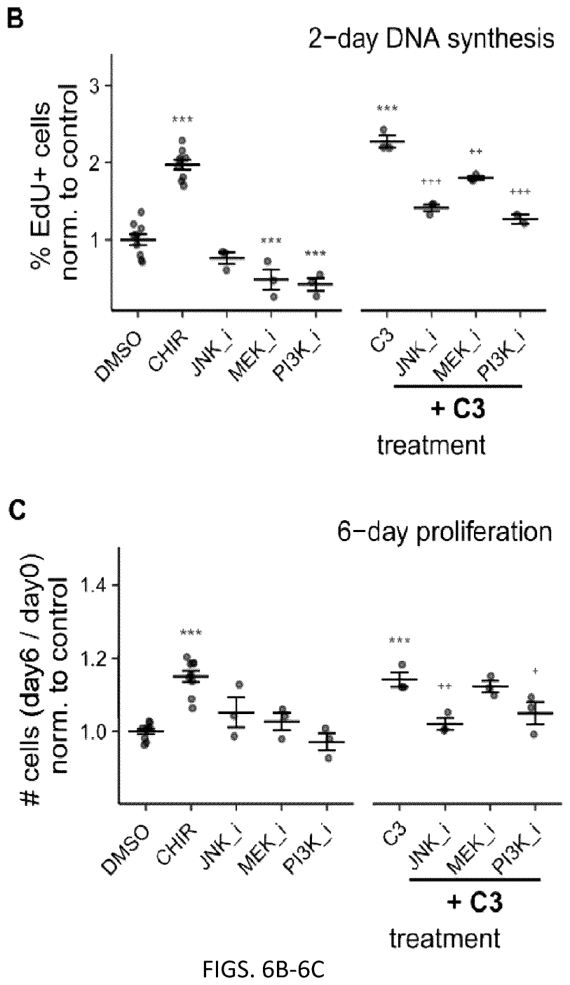

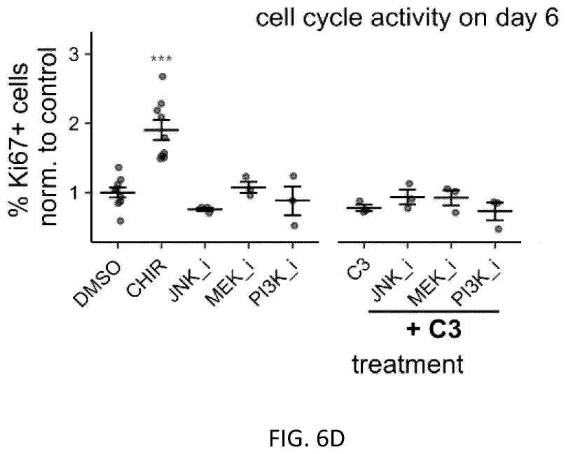

We next aimed to integrate the common proteomic signatures to downstream transcriptional regulators that drive cardiomyocyte proliferation. We sought to find causal relationships linking the set of the top 15 transcriptional regulators predicted from the RNAseq, 17 differentially expressed/activated proteins from the RPPA, and additional factors regulating proliferation confirmed by additional experimentation with the lead compounds. To reconstruct a directed signaling network, we used the SIGNOR database of manually-annotated causal relationships [35, 36] and the k shortest path algorithm from PATHLINKER [37], with RTKs as source nodes and transcription factors as target nodes (FIG. 6A). The overall direction of the proteomic responses measured by the RPPA and RTK arrays across all four compounds were mapped onto network nodes. The integrated molecular network suggested compounds activate ErbB2 and IGF1R pathways leading to the phosphorylation of RB1 and activation of E2F via the PI3K/AKT pathway. This would be expected to initiate cell cycle progression into S-phase, while inhibiting proteins associated with arresting or inhibiting the cell cycle such as CDKN1B. The network also predicts canonical RTK pathways, plus INK, MEK, and PI3K, play an important role in mediating compound-induced proliferation.

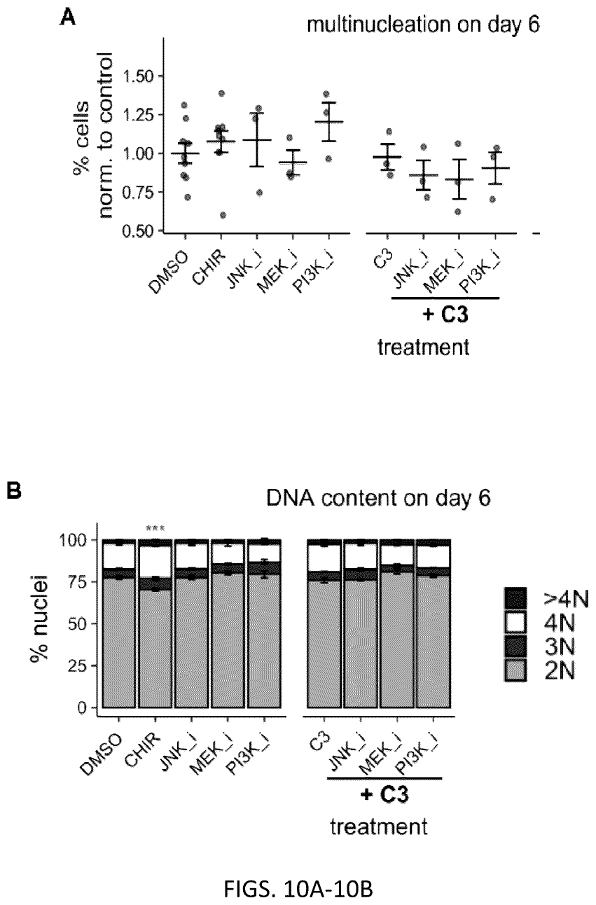

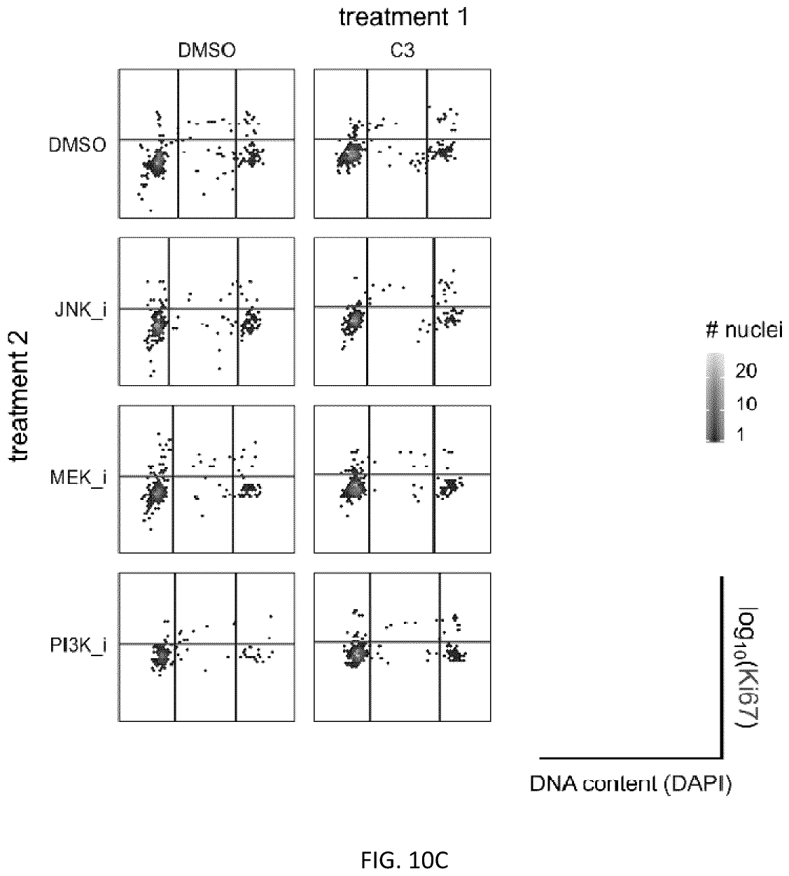

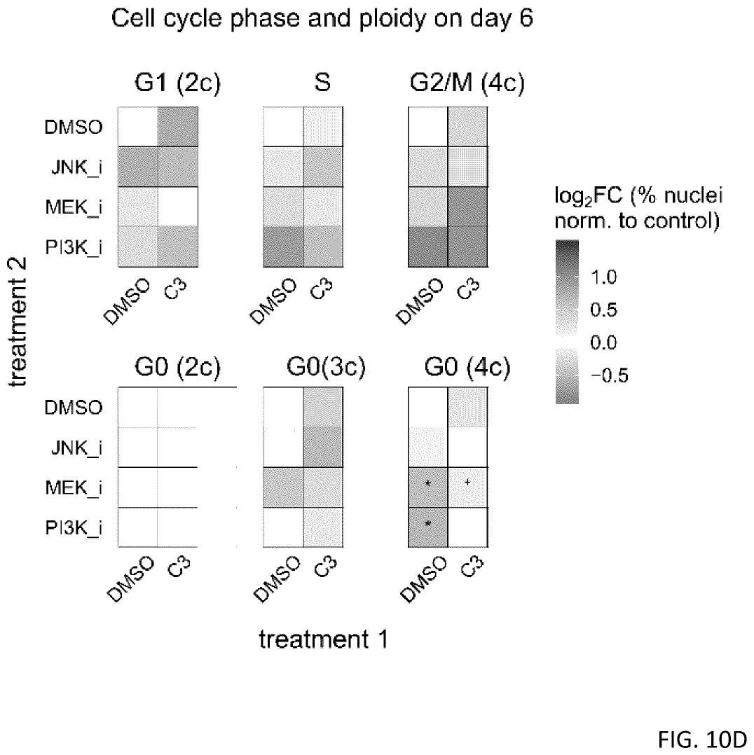

We decided to further investigate MAPK and PI3K signaling pathways, which are known as key intermediates in RTK signal transduction and supported by our integrated network. We tested small molecule inhibitors targeting INK, MEK, and PI3K in unstimulated and stimulated hiPSC-CMs. We used the same set of high-content assays to measure DNA synthesis at two days and cell division, multinucleation, DNA content, cell cycle phase distributions, and nuclear ploidy at six days post treatment with inhibitors. We found that inhibiting JNK with 10 μM SP600125, MEK with 10 μM PD98059, and PI3K with 10 μM LY294002 attenuated C3 induction of DNA synthesis at 48 h (FIG. 6B). Inhibition of MEK and PI3K in unstimulated hiPSC-CMs resulted in modest decreases in EdU incorporation (FIG. 6B). By day 6, only INK and PI3K inhibition attenuated C3 induced proliferation, with none of the three kinase inhibitors affecting proliferation in unstimulated hiPSC-CMs (FIG. 6C). Consistent with results from the RTK experiments, the overall increase in cell cycle activity was not sustained for 6 days (FIG. 6D). The anti-proliferative effects observed with JNK and PI3K inhibition in stimulated hiPSC-CMs did not correlate with increased multinucleation, G0 intermediate DNA content, or G0 tetraploidy (FIG. 10), suggesting that VEGF receptor regulation of cardiomyocyte endoreplication is not mediated via these kinases. Together, these results suggest that C3, which targets the ALK5 pathway, converges on INK, MEK, and PI3K pathways to regulate DNA synthesis and JNK and PI3K to complete cell division.

### DISCUSSION

Pharmacological manipulation of signaling pathways to stimulate proliferation of endogenous cardiomyocytes is a promising therapeutic strategy for cardiac regeneration. Further, cardiomyocyte binucleation and polyploidization have been linked to a loss of proliferative and regenerative potential [9, 10, 28, 38]. In this study, we used a systems biology approach combining phenotypic, transcriptomic, and proteomic data to identify core mechanisms regulating hiPSC-CMs proliferation. Based on hits from a previous high-content screen of a small molecule library [13], we selected five lead compounds with diverse putative targets for molecular profiling. We aimed to test whether multiple signals converge on common mechanisms to regulate cell cycle progression. Using high-throughput RNA sequencing and functional proteomic arrays, we identified a common set of transcriptional and proteomic responses mediating compound-induced proliferation and endoreplication. Integration of this multi-omic signature revealed a directed molecular network of canonical RTK pathways that were activated by multiple pro-proliferative compounds.

Characterizing mechanisms for cell cycle progression in cardiomyocytes beyond classical DNA synthesis and cell cycle markers is critical for identifying therapeutic agents that stimulate proliferation of mononucleated diploid cardiomyocytes. In this study, we applied a high-content live-cell proliferation assay [13] to comprehensively assess the effects of pharmacological perturbations on proliferation, multinucleation, polyploidization, and cell cycle activity in hiPSC-CMs. We confirmed that five hits from our previous study stimulated proliferation while maintaining or enhancing the populations of mononucleated cells and diploid nuclei. Testing multiple functional analogs to each of the lead compounds substantiated putative inhibitory targets for compound C3, implicating type 1 transforming growth factor-O receptor (ALK5), cannabinoid receptor type 1 (CB1R), and cyclooxygenase-1 and 2 (COX-1/2)/5-lipoxygenase (5-LO) as negative regulators of hiPSC-CM proliferation, respectively. We focused additional mechanistic experiments in stimulated hiPSC-CMs using inhibitors targeting receptors ALK5 (C3). Complementary genetic perturbation studies can further validate the targets responsible for the observed effects of these compounds.

In support of the pro-proliferative responses to these lead compounds, enrichment analysis of the transcriptome profiles identified robust changes in the expression of genes involved in cell cycle regulation. Removal of these genes from the analysis revealed an underlying common transcriptional program regulating DNA repair and kinesin pathways. DNA damage that remains unrepaired can lead to cell cycle arrest/exit, apoptosis, or polyploidization [39-41]. Cell cycle arrest in postnatal mammalian cardiomyocytes have been linked to DNA damage from oxidative stress [42] or telomere dysfunction [43]. Perturbing signaling pathways to stimulate proliferation in cardiomyocytes can have unanticipated consequences. For example, GSK3 deletion in adult mouse cardiomyocytes induced cell cycle re-entry and polyploidization followed by the activation of the DNA damage response pathway and mitotic catastrophe [44]. In contrast, the lead compounds in our study promoted proliferation without signs of toxicity or enhanced polyploidization, suggesting the compounds may be activating DNA repair mechanisms to enable canonical cell cycle progression. The precise role of DNA repair mechanisms in promoting cardiomyocyte proliferation remains to be determined.

We further investigated the role of the activated RTKs in mediating compound-induced proliferation. Studies have demonstrated ErbB2 signaling necessary and sufficient to induce DNA synthesis and proliferation without endoreplication in postnatal mouse cardiomyocytes [26, 52]. In partial agreement with these previous studies by others, we found pharmacological inhibition of ErbB2/EGFR attenuated C3 compound-induced DNA synthesis without affecting proliferation and polyploidization. This suggests that ErbB2/EGFR signaling is necessary for compounds to stimulate cell cycle reentry at G1/S transition, but not to promote later-stage cell division.

Previous studies investigating the role of VEGF signaling in cardiomyocyte proliferation have been inconclusive and contradictory [32-34]. For example, one group showed VEGF gene transfer in a pig MI model resulted in significant increases in cardiomyocyte mitotic index and the number of cardiomyocyte nuclei without cytokinesis [32]. However, another group found treatment with recombinant VEGF did not induce DNA synthesis or proliferation in vitro in neonatal rat cardiomyocytes [34]. In our study, we found that while pharmacological inhibition of VEGF receptors did not affect compound-induced DNA synthesis, VEGF receptor signaling mediated the pro-proliferative effects exerted by the compounds in hiPSC-CMs. Additional analysis revealed inhibiting VEGF receptor signaling induced binucleation, intermediate DNA content, and polyploidy in both unstimulated and stimulated hiPSC-CMs, suggesting increased expression of VEGF receptors protects against polyploidization and aberrant mitosis. Further experiments are needed to discern whether the increased intermediate DNA content results from increased aneuploidy.

Our reconstructed directed molecular network focused on connecting common proteomic and transcriptomic signatures downstream of the activated RTKs. Analysis of the network suggested canonical RTK signaling mediators INK, MEK/ERK, and PI3K/AKT were involved in regulating compound-induced proliferation. JNK [53, 54], MEK/ERK [1, 55, 56], and PI3K/AKT [28, 57-59] have each been implicated in mediating cell cycle reentry, linking multiple pathways to cardiomyocyte proliferation. However, other studies discovered agents that stimulate cardiomyocyte proliferation independent of PI3K/AKT and MEK/ERK [12, 38, 60]. Therefore, we tested selective inhibitors to determine the requirement for mechanistic roles of these kinases in mediating proliferation and endoreplication. We found that pharmacological inhibition of all three kinases attenuated C3-induced DNA synthesis. However, only INK and PI3K activities were necessary for the compounds to induce bona-fide proliferation of hiPSC-CMs. Future work could test additional kinase inhibitors to rule out concerns about their limited specificity. While RTK signaling pathways are known to converge on MAPK and PI3K modules, none of the kinases we investigated were found to mediate VEGF receptor regulation of cardiomyocyte endoreplication. Additional studies are needed to identify how the VEGF receptor signaling is linked to endoreplication and other mechanisms induced by the compounds. For example, all four compounds induced expression of TAZ (FIG. 4D), a member of the Hippo complex that is a well-established hub of cardiomyocyte proliferation [61]. Given the relative immaturity of hiPSC-CMs, future studies will be needed to test these compounds and proliferative mechanisms in adult CMs.

In summary, we identified a core set of mechanisms by which multiple compounds regulate cell cycle progression of hiPSC-CMs. Our study identified multiple putative targets that negatively regulated proliferation without endoreplication, including ALK5 and CB1R. We also discovered the lead compounds that collectively activated RTKs (ErbB2/EGFR and VEGF receptors) and activated downstream JNK and PI3K pathways to regulate DNA synthesis, endoreplication, and cell division in hiPSC-CMs.

### Methods

### Materials Availability

The compounds corresponding to treatments named in the manuscript (C3/RepSox, CHIR99021 (aka CHIR), PD98059, LY294002, SP600125, lapatinib, axitinib, linsitinib, C3a/SB525334, C3c/galunisertib, C4a/taranabant, C4d/rimonabant, C5a/indometacin, C5c/celecoxib, and C5e/SC560) are commercially available compounds.

### Data and Code Availability

RNA-seq data have been deposited at GEO under accession GSE268693 and are publicly available as of the date of publication. RPPA data have been deposited at FigShare and are publicly available as of the date of publication.

### Experimental Model and Study Participants Detail

### Cell Culture

ICell Cardiomyocytes (Cellular Dynamics International) were seeded in 96-well or 384-well Corning CellBind plates at approximately 2.5×10⁴ to 3.5×10⁴ cells/cm². Cells were cultured for 2 days in iCell Cardiomyocytes Plating Medium (Cellular Dynamics International) supplemented with 1% Penicillin/Streptomycin (P/S; Gibco) followed by an additional 4 days in iCell Cardiomyocytes Maintenance Medium (Cellular Dynamics International) supplemented with 1% P/S. For all experiments, cells were first serum starved for 4 h and then treated in William's E medium without phenol red (Gibco) supplemented with Primary Hepatocyte Cell Maintenance Cocktail B (Gibco) containing P/S, ITS+, GlutaMAX, and HEPES. Cells were cultured for all experiments in a humidified environment at 37° C. with 5% CO₂.

### Method Details

### DNA Synthesis Assay

EdU incorporation and labeling with copper catalyzed click chemistry was used to measure DNA synthesis. Cells were treated in the presence of 1 μM EdU (Invitrogen) for 48 h, and subsequently fixed and permeabilized prior to incubation with the EdU click chemistry reaction mix—100 mM Tris HCL pH 7.0 (Gentrox), 4 mM CuSO₄ (Sigma), 100 mM ascorbic acid (Sigma), and 5 μM Alexa Fluor 546 Azide (Click Chemistry Tools). After labelling EdU, cells were stained with anti-Cardiac Troponin T (Abcam) and DAPI (Sigma).

### Live-Cell Proliferation Assay

To track the change in the number of live cells over 6 days, the cells were stained with a non-toxic concentration (0.02 μg/mL) of Hoechst 33342 (Invitrogen) and imaged immediately after treatment [11]. Treatment medium was replaced on day 3 to replenish nutrients, and the cells were stained with Hoechst 33342 and imaged again on day 6. Images of the same fields of view were acquired at both initial and final timepoints by aligning the multi-well plates using distinct tracking markers in the upper-left and bottom-right most wells. After acquiring live-cell images on day 6, cells were fixed and stained with anti-Cardiac Troponin T, anti-Ki67 (Invitrogen), and DAPI for subsequent binucleation, DNA content, cell-cycle phase, and nuclear ploidy analyses.

### Immunostaining

Cells were fixed with 4% paraformaldehyde (Ted Pella) for 15 min, permeabilized with 0.2% TritonX-100 (MP Biomedicals) for 10 min at room temperature (RT), blocked with 5% bovine serum albumin (Sigma) for 1 h at RT, and incubated with primary antibodies (see Table 1) overnight at 4° C. Cells were then rinsed with 1×PBS (Gibco) three times, blocked again with 2% goat or donkey serum (Invitrogen) for 1 h at RT, and incubated with secondary antibodies for 1 h. After antibody staining, cells were rinsed with 1×PBS three times and incubated with DAPI for 10 min at RT.

### Automated Image Acquisition

Stained cells were imaged using either the automated Olympus IX81 inverted microscope with motorized functions and a 10×UPlanSApo 0.4 NA objective or the Operetta CLS high-content imaging system (Perkin-Elmer) with a 10×0.3NA objective. For imaging with the Olympus system, multi-well imaging pipelines were developed and executed using MetaMorph software (Olympus) to automate image acquisition and stitching of multi-channel 2×2 mosaics with 10% overlap. For the Operetta system, one field of view was acquired per well using the Harmony high-content imaging software (Perkin-Elmer).

### Automated Image Analysis

Custom image analysis and processing pipelines were developed and implemented in MATLAB to quantify the number of nuclei/cells, percent EdU positive objects, percent Ki67 positive objects, multinucleation, DNA content, cell cycle phases, and ploidy. Code is freely available (https://github.com/saucermanlab/Woo_JMCC_PMID30597148).

Segmentation—Nuclei stained with either Hoechst 33342 or DAPI were segmented using a pipeline adapted from our previous works in high-content imaging [13, 62] and functions from MATLAB's Image Processing Toolbox. Briefly, image data was smoothed with an adaptive low-pass Wiener filter and transformed to a binary mask using a global threshold based on the mode and variance of the intensity values. Clumps of nuclei were separated by applying the watershed algorithm to the distance transform map of the complement of the binary mask.

Classification—The binary segmented mask and background subtracted images were used to calculate the mean intensities of Hoechst 33342, DAPI, Cardiac-Troponin T, EdU, and Ki67 staining for each segmented object. Nuclei with Hoechst 33342 or DAPI mean intensity values greater than 5 standard deviations above the population mean were excluded from all counts. Marker positivity thresholds were determined using Rosin's unimodal thresholding algorithm [63] on the mean intensity values for each segmented object.

Pharmacology Metrics—Based on concentration-dependent responses of changes in cell number, we quantified area under the curve (AUC) as the sum of the relative changes in cell number across the five concentrations of compound. As a measure of potency, we performed cubic B-spline interpolation and then calculated the minimum concentration needed to achieve 50% of the maximum effect.

Multinucleation Analysis—Multinucleated cells were identified based on the distance between neighboring nuclei. This distance threshold was determined using Cardiac-Troponin T staining to visualize cell borders. A morphological closing operation with a circular structuring element was applied to the binary segmented mask to merge and classify nuclei in multinucleated cells.

Ploidy and Cell Cycle Phase Analysis—Our previous k-means clustering approach for measuring DNA content [13] was modified to identify nuclei with intermediate DNA content (3c) in addition to the 2c, 4c, and >4c nuclei. The updated algorithm was adapted from classical flow-cytometry DNA content analysis approaches [64, 65] using the histogram of integrated intensity measurements of DAPI or Hoechst 33342 segmented objects. Briefly, gaussian distribution functions were fitted to the 2c and 4c peaks of DNA content, and the 3c population was estimated using a uniform distribution with height set to the average binned frequencies between the peaks. Ki67 staining was used to discriminate between actively cycling nuclei or nuclei in G0. Combining DNA content analysis and Ki67 positivity enabled classification of nuclei into 6 cell cycle phase and ploidy states—G1 (2c), S (3c), G2/M (4c), G0 (2c), G0 with intermediate DNA content (3c), and G0 tetraploid (4c).

### RNA Sequencing

Total RNA was isolated from hiPSC-CMs treated for 24 h using the mirVana microRNA Isolation Kit with phenol (Invitrogen) according to the manufacturer's guidelines. Each sample was run with 3 biological replicates. RNA integrity was assessed using the Agilent 2100 Bioanalyzer with the Agilent RNA 6000 Pico Kit by the University of Virginia's Genomics Core. All samples had RIN values ranging from 9.9-10.0. RNA sequencing libraries were generated and indexed using 200 ng of total RNA and the TruSeq Stranded mRNA LT Sample Prep Kit-Set B (Illumina) following the protocol provided by the manufacturer. Resulting libraries were then assessed using the Agilent TapeStation 4200 (Agilent Technologies) and quantified by Qubit 2.0 (Thermo Fisher Scientific). The indexed libraries were pooled at equal molar concentrations and paired-end sequenced with 75 bp per read on the Illumina NextSeq 500 system using NextSeq 500 High Output 150 cycle cartridges (Illumina).

### RNA-Seq Analysis

Sequence read quality was assessed using the FastQC software (http://www.bioinformatics. babraham.ac.uk/projects/fastqc). All samples at all positions had Phred scores greater than 30. Paired-end reads were mapped to the human reference genome hg38 using the Bowtie2 aligner [66] and gene-level counts were quantified using the featureCounts program [67]. Genes with low counts were removed before normalizing by TMM. Normalized gene counts were voom transformed for principal component analysis. DESeq2 [68] was used for differential gene expression analysis between compound-treatment and the negative control with an FDR significance level of 0.05. Pathway enrichment of differentially expressed gene sets was performed using EnrichR API platform with the Reactome 2016 database [17, 18]. Transcription factor enrichment analysis was performed using ChEA3 API platform with the ARCHS4 co-expression database [19].

### Phospho-RTK Array

Cells were seeded in 60 mm Corning CellBind dishes, cultured for 6 days, serum starved for 4 h, and treated with compounds for 12 h with 3 replicates. At the end of the experiment, cells were treated with 1 mM peroxyvanadate (1 mM NaVO₃, 0.1 μg/mL Catalase, 0.003% H₂O₂) for 15 min prior to cell lysis. Cells were then rinsed with ice-cold 1×PBS and lysed on ice for 15 min with lysis buffer (50 mM Tris-HCl pH 7.5, 150 mM NaCl, 0.1 mM EDTA, 1 mM DTT, 1 mM NaVO3, 0.5% TritonX-100) containing protease inhibitor cocktail (Sigma). Cell lysates were mechanically homogenized using 28-gauge syringes, spun down, and adjusted to equivalent concentrations. The Proteome Profiler Human Phospho-RTK Array Kit (R&D Systems) was used to measure phosphorylation activity of 49 RTKs. Briefly, the arrays with printed anti-RTK antibodies were blocked for 1 hour and incubated with 38.8 μg of total protein lysate overnight at 4° C. The arrays were then washed and incubated with anti-phospho-tyrosine antibodies conjugated to HRP and detected via chemiluminescence. Array films were scanned and Image Studio software (LI-COR) was used to quantify intensities of each spot. Intensity values were background subtracted and normalized to the positive control spots on each array.

### Reverse Phase Protein Array

The same cell lysates prepared for the RTK array were also used for the RPPA to measure protein and phospho-protein expression levels of over 250 proteins. The RPPA was performed and quantified by the University of Texas MD Anderson Cancer Center's Functional Proteomics RPPA Core. The MD Anderson platform included 240 total protein and 65 phospho-protein probes. Briefly, serial dilutions of lysates were printed on nitrocellulose-coated slides and each slide was probed with a different antibody. Signals were detected by tyramide signal amplification and DAB colorimetric reaction systems. Images of scanned slides were quantified by Array-Pro Analyzer. Relative protein levels were determined using the SuperCurve program (https://bioinformatics.mdanderson.org/public-software/supercurve/) and normalized to correct for protein loading and antibody variation. Linear models with the empirical Bayes [69] approach via the limma package [70] was used to assess differential expression in the RPPA between compounds and the DMSO negative control with an adjusted p-value significance level of 0.1.

### Network Integration

To reconstruct a directed network integrating the multi-omic signature, we used SIGNOR's manually-annotated database of causal relationships linking biological factors [35, 36]. We filtered the database to include relationships involving the set of common factors and their first neighbors, and excluded relationships with confidence scores less than 0.2. Next, we used the k shortest path algorithm from PATHLINKER [37], with k=20, to identify the pathways linking ErBB2, IGF1R, or VEGFR2 to the set of top 15 predicted transcription factors overlapping the set of proteins included in the filtered relationships. The shortest paths networks for each RTK were then merged and redundant relationships were removed. To visualize the overall proteomic responses on the network, each protein in the RPPA or RTK was assigned+1 for upregulated, −1 for downregulated, or 0 for not significant. We then mapped the sum of the signal direction onto the network nodes. For example, a node with value of +4 indicates the protein was upregulated across all four compounds.

### Quantification and Statistical Analysis

### Statistical Analysis for Phenotypic Experiments

Phenotypic experiments were performed with 2-4 replicates for perturbation groups and 6-24 replicates for control groups. Measurements from experiments requiring multiple multi-well plates were normalized to the negative control wells in each plate. All statistical tests were implemented in R using the stats and multcomp v1.4-13 packages in R v3.6.1. Statistical significance was determined for experiments with multiple treatments using a one-way ANOVA with post-hoc test for multiple comparison correction (Dunnett, Benjamini-Hotchberg). Plots were generated in R using ggplot2 v3.2.1, ComplexHeatmap v2.0.0, cowplot v1.0.0, and VennDiagram v1.6.20. Error bars represent mean±s.e.m. The number of replicates, statistical tests, and significance levels for phenotypic experiments are noted in the figure legends.

TABLE 1
Key Resources

| | | |
|---|---|---|
| REAGENT | SOURCE | CATALOG # |

Antibodies and Stains

| | | |
|---|---|---|
| Rabbit polyclonal anti- | Abcam | cat# ab45932; |
| Cardiac Troponin T | | RRID AB_956386 |
| Rat monoclonal anti-Ki67 | Invitrogen | cat# 14-5698-82; |
| | | RRID AB_10854564 |
| DAPI | Sigma | cat# D9542-5MG |
| Hoechst 33342 | Invitrogen | cat# H3570 |

Chemicals

| | | |
|---|---|---|
| EdU | Invitrogen | cat# A10044 |
| CHIR99021 | Sigma-Aldrich | cat# SML1046 |
| PD98059 | TOCRIS | cat# 1213 |
| Lapatinib | TOCRIS | cat #6811 |
| LY294002 | Selleckchem | cat# S1105 |
| SP600125 | Selleckchem | cat# S1460 |
| Axitinib | Selleckchem | cat# S1005 |
| Linsitinib (OSI-906) | Selleckchem | cat# S1091 |
| AF 546 Azide (for EdU click | Click Chemistry Tools | cat# 1283-1 |
| chemistry) | | |
| William's E media, no | Gibco | cat# A1217601 |
| phenol red | | |
| Primary Hepatocyte Cell | Gibco | cat# CM4000 |
| Maintenance Cocktail B | | |
| iCell Cardiomyocytes Plating | Cellular Dynamics | cat# CMM-100-110-005 |
| Medium | International | |
| iCell Cardiomyocytes Maintenance | Cellular Dynamics | cat# CMM-100-120-005 |
| Medium | International | |
| Penicillin/Streptomycin | Gibco | cat# 15140122 |
| Protease inhibitor cocktail | Sigma | cat# P8340 |

Commercial Assays

| | | |
|---|---|---|
| mirVana microRNA Isolation Kit, | Invitrogen | cat# AM1560 |
| with phenol | | |
| Proteome Profiler Human | R&D Systems | cat# ARY001B |
| Phospho-RTK Array Kit | | |

Experimental Models: Cell Lines

| | | |
|---|---|---|
| iCell Human iPSC-derived | Cellular Dynamics | cat# CMC-100-010-001 |
| cardiomyocytes | International | |

| | | |
|---|---|---|
| RESOURCE | SOURCE | IDENTIFIER |

Software and Algorithms

| | | |
|---|---|---|
| MetaMorph | Olympus | |
| Harmony | Perkin-Elmer | |
| MATLAB R2017B | Mathworks | |
| R v3.6.1 | R Project | RRID: SCR_001905 |
| Fast QC | | RRID: SCR_014583 |
| Bowtie2 | Langmead et al., 2012 | http://bowtie- |
| | | bio.sourceforge.net/ |
| | | bowtie2/index.shtml |
| featureCounts | Liao et al., 2014 | RRID: SCR_012919 |
| DESeq2 v1.24.0 | Love et al. 2014 | RRID: SCR_015687 |
| LIMMA v3.40.2 | Ritchie et al., 2015 | https://bioconductor.org/ |
| | | packages/release/bioc/html/ |
| | | limma.html |
| EnrichR | Chen et al., 2013 | https://maayanlab.cloud/ |
| | | Enrichr/ |
| ChEA3 | Keenan et al., 2019 | https://amp.pharm.mssm.edu/ |
| | | chea3/ |
| ggplot2 v3.2.1 | | RRID: SCR_014601 |
| | | http://bioconductor.org/ |
| ComplexHeatmap v2.0.0 | Gu et al., 2016 | packages/release/bioc/html/ |
| | | ComplexHeatmap.html |
| multcomp v1.4-13 | Hothorn et al., 2008 | http://multcomp.R-forge.R- |
| | | project.org |

### REFERENCES

[1] Uosaki H, Magadum A, Seo K, et al. Identification of chemicals inducing cardiomyocyte proliferation in developmental stage-specific manner with pluripotent stem cells. Circ Cardiovasc Genet 2013; 6: 624-633.
[2] Eulalio A, Mano M, Dal Ferro M, et al. Functional screening identifies miRNAs inducing cardiac regeneration. Nature 2012; 492: 376-381.
[3] Diez-Cuñado M, Wei K, Bushway P J, et al. miRNAs that Induce Human Cardiomyocyte Proliferation Converge on the Hippo Pathway. Cell Rep 2018; 23: 2168-2174.
[4] Titmarsh D M, Glass N R, Mills R J, et al. Induction of Human iPSC-Derived Cardiomyocyte Proliferation Revealed by Combinatorial Screening in High Density Microbioreactor Arrays. Sci Rep 2016; 6: 24637.
[5] Mills R J, Parker B L, Quaife-Ryan G A, et al. Drug Screening in Human PSC-Cardiac Organoids Identifies Pro-proliferative Compounds Acting via the Mevalonate Pathway. Cell Stem Cell 2019; 24: 895-907.e6.
[6] Patterson M, Barske L, Van Handel B, et al. Frequency of mononuclear diploid cardiomyocytes underlies natural variation in heart regeneration. Nat Genet. Epub ahead of print 7 Aug. 2017. DOI: 10.1038/ng.3929.
[7] Bensley J G, De Matteo R, Harding R, et al. Three-dimensional direct measurement of cardiomyocyte volume, nuclearity, and ploidy in thick histological sections. Scientific Reports 2016; 6: 23756.
[8] Bergmann O, Zdunek S, Felker A, et al. Dynamics of Cell Generation and Turnover in the Human Heart. Cell 2015; 161: 1566-1575.
[9] Windmueller R, Leach J P, Babu A, et al. Direct Comparison of Mononucleated and Binucleated Cardiomyocytes Reveals Molecular Mechanisms Underlying Distinct Proliferative Competencies. Cell Rep 2020; 30: 3105-3116.e4.
[10] González-Rosa J M, Sharpe M, Field D, et al. Myocardial polyploidization creates a barrier to heart regeneration in zebrafish. Dev Cell 2018; 44: 433-446.e7.
[11] Woo L A, Tkachenko S, Ding M, et al. High-content phenotypic assay for proliferation of human iPSC-derived cardiomyocytes identifies L-type calcium channels as targets. J Mol Cell Cardiol2019; 127: 204-214.
[12] Magadum A, Ding Y, He L, et al. Live cell screening platform identifies PPAR6 as a regulator of cardiomyocyte proliferation and cardiac repair. Cell Research 2017; 27: 1002-1019.
[13] Woo L A, Tkachenko S, Ding M, et al. High-content phenotypic assay for proliferation of human iPSC-derived cardiomyocytes identifies L-type calcium channels as targets. J Mol Cell Cardiol2018; 127: 204-214.
[14] Sharma A, Burridge P W, McKeithan W L, et al. High-throughput screening of tyrosine kinase inhibitor cardiotoxicity with human induced pluripotent stem cells. Science Translational Medicine; 9. Epub ahead of print 15 Feb. 2017. DOI: 10.1126/scitranslmed.aaf2584.
[15] Berenbaum M C. What is synergy?Pharmacol Rev 1989; 41: 93-141.
[16] Foucquier J, Guedj M. Analysis of drug combinations: current methodological landscape. Pharmacol Res Perspect; 3. Epub ahead of print June 2015. DOI: 10.1002/prp2.149.
[17] Chen E Y, Tan C M, Kou Y, et al. Enrichr: interactive and collaborative HTML5 gene list enrichment analysis tool. BMC Bioinformatics 2013; 14: 128.
[18] Kuleshov M V, Jones M R, Rouillard A D, et al. Enrichr: a comprehensive gene set enrichment analysis web server 2016 update. Nucleic Acids Res 2016; 44: W90-97.
[19] Keenan A B, Torre D, Lachmann A, et al. ChEA3: transcription factor enrichment analysis by orthogonal omics integration. Nucleic Acids Res 2019; 47: W212-W224.
[20] Ebelt Henning, Hufnagel Nadine, Neuhaus Petra, et al. Divergent Siblings. Circulation Research 2005; 96: 509-517.
[21] Ebelt H, Zhang Y, Kampke A, et al. E2F2 expression induces proliferation of terminally differentiated cardiomyocytes in vivo. Cardiovasc Res 2008; 80: 219-226.
[22] Sengupta A, Kalinichenko V V, Yutzey K E. FoxO1 and FoxM1 transcription factors have antagonistic functions in neonatal cardiomyocyte cell-cycle withdrawal and IGF1 gene regulation. Circ Res 2013; 112: 267-277.
[23] Bolte C, Zhang Y, Wang I-C, et al. Expression of Foxml transcription factor in cardiomyocytes is required for myocardial development. PLoS ONE 2011; 6: e22217.
[24] Korver W, Schilham M W, Moerer P, et al. Uncoupling of S phase and mitosis in cardiomyocytes and hepatocytes lacking the winged-helix transcription factor Trident. Curr Biol 1998; 8:1327-1330.
[25] Grundl M, Walz S, Hauf L, et al. Interaction of YAP with the Myb-MuvB (MMB) complex defines a transcriptional program to promote the proliferation of cardiomyocytes. PLOS Genetics 2020; 16: e1008818.
[26]D'Uva G, Aharonov A, Lauriola M, et al. ERBB2 triggers mammalian heart regeneration by promoting cardiomyocyte dedifferentiation and proliferation. Nat Cell Biol 2015; 17: 627-638.
[27] Honkoop H, de Bakker D E, Aharonov A, et al. Single-cell analysis uncovers that metabolic reprogramming by ErbB2 signaling is essential for cardiomyocyte proliferation in the regenerating heart. Elife; 8. Epub ahead of print 23 Dec. 2019. DOI: 10.7554/eLife.50163.
[28] Bersell K, Arab S, Haring B, et al. Neuregulin1/ErbB4 Signaling Induces Cardiomyocyte Proliferation and Repair of Heart Injury. Cell 2009; 138: 257-270.
[29] Huang Y, Harrison M R, Osorio A, et al. Igf Signaling is Required for Cardiomyocyte Proliferation during Zebrafish Heart Development and Regeneration. PLoS One; 8. Epub ahead of print 26 Jun. 2013. DOI: 10.1371/journal.pone.0067266.
[30] McDevitt T C, Laflamme M A, Murry C E. Proliferation of cardiomyocytes derived from human embryonic stem cells is mediated via the IGF/PI 3-kinase/Akt signaling pathway. Journal of Molecular and Cellular Cardiology 2005; 39: 865-873.
[31] Jennbacken K, Wigberg F, Karlsson U, et al. Phenotypic Screen with the Human Secretome Identifies FGF16 as Inducing Proliferation of iPSC-Derived Cardiac Progenitor Cells. Int J Mol Sci; 20. Epub ahead of print 30 Nov. 2019. DOI: 10.3390/ijms20236037.
[32] Laguens R, Meckert P C, Janavel G V, et al. Entrance in mitosis of adult cardiomyocytes in ischemic pig hearts after plasmid-mediated rhVEGF 165 gene transfer. Gene Therapy 2002; 9: 1676-1681.
[33] Xu X H, Xu J, Xue L, et al. VEGF attenuates development from cardiac hypertrophy to heart failure after aortic stenosis through mitochondrial mediated apoptosis and cardiomyocyte proliferation. Journal of Cardiothoracic Surgery 2011; 6: 54.
[34] Zentilin L, Puligadda U, Lionetti V, et al. Cardiomyocyte VEGFR-1 activation by VEGF-B induces compensatory hypertrophy and preserves cardiac function after myocardial infarction. The FASEB Journal 2010; 24: 1467-1478.
[35] Perfetto L, Briganti L, Calderone A, et al. SIGNOR: a database of causal relationships between biological entities. Nucleic Acids Res 2016; 44: D548-554.
[36] Licata L, Lo Surdo P, Iannuccelli M, et al. SIGNOR 2.0, the SIGnaling Network Open Resource 2.0: 2019 update. Nucleic Acids Res 2020; 48: D504-D510.
[37] Ritz A, Poirel C L, Tegge A N, et al. Pathways on demand: automated reconstruction of human signaling networks. NPJ Syst Biol Appl 2016; 2: 16002.
[38] Kuhn B, del Monte F, Hajjar R J, et al. Periostin induces proliferation of differentiated cardiomyocytes and promotes cardiac repair. Nature Medicine 2007; 13: 962-969.
[39] Jackson S P, Bartek J. The DNA-damage response in human biology and disease. Nature 2009; 461: 1071-1078.
[40] Ivanov A, Cragg M S, Erenpreisa J, et al. Endopolyploid cells produced after severe genotoxic damage have the potential to repair DNA double strand breaks. Journal of Cell Science 2003; 116: 4095-4106.
[41] Baus F, Gire V, Fisher D, et al. Permanent cell cycle exit in G2 phase after DNA damage in normal human fibroblasts. EMBO J 2003; 22: 3992-4002.
[42] Puente B N, Kimura W, Muralidhar S A, et al. The oxygen-rich postnatal environment induces cardiomyocyte cell-cycle arrest through DNA damage response. Cell 2014; 157: 565-579.
[43] Aix E, Gutiérrez-Gutiérrez Ó, Sänchez-Ferrer C, et al. Postnatal telomere dysfunction induces cardiomyocyte cell-cycle arrest through p21 activation. J Cell Biol 2016; 213: 571-583.
[44] Zhou J, Ahmad F, Parikh S, et al. Loss of Adult Cardiac Myocyte GSK-3 Leads to Mitotic Catastrophe Resulting in Fatal Dilated Cardiomyopathy. Circ Res 2016; 118: 1208-1222.
[45] Dalton G D, Peterson L J, Howlett A C. CB₁ cannabinoid receptors promote maximal FAK catalytic activity by stimulating cooperative signaling between receptor tyrosine kinases and integrins in neuronal cells. Cell Signal 2013; 25: 1665-1677.
[46] Dalton G D, Howlett A C. Cannabinoid CB1 receptors transactivate multiple receptor tyrosine kinases and regulate serine/threonine kinases to activate ERK in neuronal cells. Br J Pharmacol 2012; 165: 2497-2511.
[47] Rubovitch V, Gafni M, Sarne Y. The involvement of VEGF receptors and MAPK in the cannabinoid potentiation of Ca2+flux into N18TG2 neuroblastoma cells. Brain Res Mol Brain Res 2004; 120: 138-144.
[48] Laprairie R B, Bagher A M, Kelly M E M, et al. Type 1 cannabinoid receptor ligands display functional selectivity in a cell culture model of striatal medium spiny projection neurons. J Biol Chem 2014; 289: 24845-24862.
[49] Khajehali E, Malone D T, Glass M, et al. Biased Agonism and Biased Allosteric Modulation at the CB1 Cannabinoid Receptor. Mol Pharmacol 2015; 88: 368-379.
[50] Mukhopadhyay S, Howlett A C. Chemically distinct ligands promote differential CB1 cannabinoid receptor-Gi protein interactions. Mol Pharmacol 2005; 67: 2016-2024.
[51] Plowright A T, Nilsson K, Antonsson M, et al. Discovery of agonists of cannabinoid receptor 1 with restricted central nervous system penetration aimed for treatment of gastroesophageal reflux disease. J Med Chem 2013; 56: 220-240.
[52] Ma H, Yin C, Zhang Y, et al. ErbB2 is required for cardiomyocyte proliferation in murine neonatal hearts. Gene 2016; 592: 325-330.
[53] Peng X, Fan S, Tan J, et al. Wnt2bb Induces Cardiomyocyte Proliferation in Zebrafish Hearts via the jnkl/c-jun/crebl Pathway. Front Cell Dev Biol; 8. Epub ahead of print 2020. DOI: 10.3389/fcell.2020.00323.
[54] Hammoud L, Burger D E, Lu X, et al. Tissue inhibitor of metalloproteinase-3 inhibits neonatal mouse cardiomyocyte proliferation via EGFR/JNK/SP-1 signaling. American Journal of Physiology-Cell Physiology 2009; 296: C735-C745.
[55] Bassat E, Mutlak Y E, Genzelinakh A, et al. The extracellular matrix protein agrin promotes heart regeneration in mice. Nature 2017; 547: 179-184.
[56] Novoyatleva T, Diehl F, van Amerongen M J, et al. TWEAK is a positive regulator of cardiomyocyte proliferation. Cardiovasc Res 2010; 85: 681-690.
[57] Engel F B, Schebesta M, Duong M T, et al. p38 MAP kinase inhibition enables proliferation of adult mammalian cardiomyocytes. Genes Dev 2005; 19: 1175-1187.
[58] Lin Zhiqiang, Zhou Pingzhu, von Gise Alexander, et al. Pi3kcb Links Hippo-YAP and PI3K-AKT Signaling Pathways to Promote Cardiomyocyte Proliferation and Survival. Circulation Research 2015; 116: 35-45.
[59] Beigi F, Schmeckpeper J, Pow-anpongkul P, et al. C3orf58, a Novel Paracrine Protein, Stimulates Cardiomyocyte Cell Cycle Progression Through the PI3K-AKT-CDK7 Pathway. Circ Res; 113. Epub ahead of print 2 Aug. 2013. DOI: 10.1161/CIRCRESAHA.113.301075.
[60] Yue Z, Chen J, Lian H, et al. PDGFR-0 Signaling Regulates Cardiomyocyte Proliferation and Myocardial Regeneration. Cell Reports 2019; 28: 966-978.e4.
[61] Heallen T, Zhang M, Wang J, et al. Hippo pathway inhibits Wnt signaling to restrain cardiomyocyte proliferation and heart size. Science (New York, N Y) 2011; 332: 458-461.
[62] Bass G T, Ryall K A, Katikapalli A, et al. Automated image analysis identifies signaling pathways regulating distinct signatures of cardiac myocyte hypertrophy. J Mol Cell Cardiol 2012; 52: 923-930.
[63] Rosin P L. Unimodal thresholding. Pattern Recognition 2001; 34: 2083-2096.
[64] Watson J V, Chambers S H, Smith P J. A pragmatic approach to the analysis of DNA histograms with a definable G1 peak. Cytometry 1987; 8: 1-8.
[65] Dean P N, Jett J H. Mathematical analysis of DNA distributions derived from flow microfluorometry. J Cell Biol 1974; 60: 523-527.
[66] Langmead B, Salzberg S L. Fast gapped-read alignment with Bowtie 2. Nat Methods 2012; 9: 357-359.
[67] Liao Y, Smyth G K, Shi W. featureCounts: an efficient general purpose program for assigning sequence reads to genomic features. Bioinformatics 2014; 30: 923-930.
[68] Love M I, Huber W, Anders S. Moderated estimation of fold change and dispersion for RNA-seq data with DESeq2. Genome Biol; 15. Epub ahead of print 2014. DOI: 10.1186/s13059-014-0550-8.
[69] Smyth G K. Linear models and empirical bayes methods for assessing differential expression in microarray experiments. Stat Appl Genet Mol Biol 2004; 3: Article3.
[70] Ritchie M E, Phipson B, Wu D, et al. limma powers differential expression analyses for RNA-sequencing and microarray studies. Nucleic Acids Res 2015; 43: e47-e47.

What is claimed is:

1. A method for inducing proliferation of a cardiomyocyte culture, the method comprising contacting the cardiomyocyte culture with a therapeutic agent selected from:

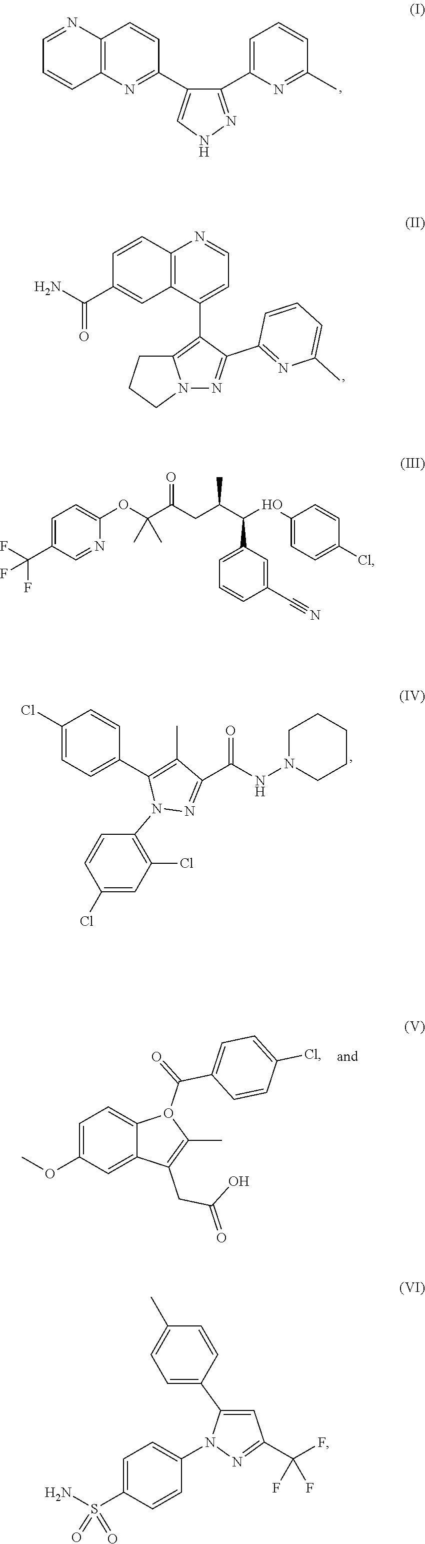

2. The method of claim 1, wherein the therapeutic agent is

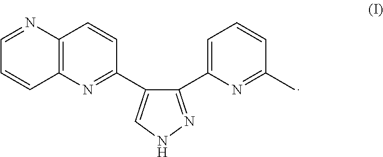 [SMILES: Cc1cccc(-c2n[nH]cc2-c2ccc3ncccc3n2)n1]

3. The method of claim 1, wherein the therapeutic agent is provided at a concentration of between about 0.1 and about 10 μM.

4. The method of claim 2, wherein 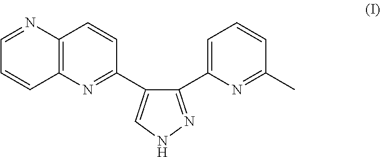 [SMILES: Cc1cccc(-c2n[nH]cc2-c2ccc3ncccc3n2)n1]
is provided at a concentration of about 3 μM.

5. The method of claim 1, wherein the cardiomyocyte culture comprises binucleated or polyploid cardiomyocytes, and wherein contacting the culture with the therapeutic agent does not increase the amount of binucleated or polyploid cardiomyocytes.

6. The method of claim 1, wherein the cardiomyocyte culture comprises primary cells or a cell line.

7. The method of claim 6, wherein the cell line is iCell cardiomyocyte cell line.

8. A method for inducing expression of at least one of VEGFR2 (KDR) and ErbB2 expression in a cell, the method comprising contacting the cell with

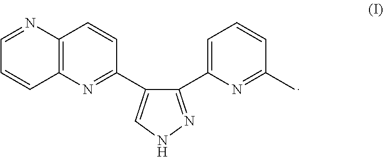 [SMILES: Cc1cccc(-c2n[nH]cc2-c2ccc3ncccc3n2)n1]

9. The method of claim 8, wherein 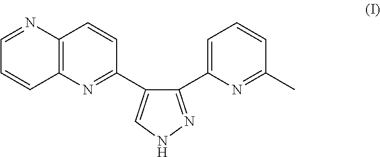 [SMILES: Cc1cccc(-c2n[nH]cc2-c2ccc3ncccc3n2)n1]
is provided at a concentration of between about 0.1 and about 10 μM.

10. The method of claim 9, wherein 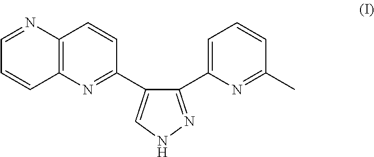 [SMILES: Cc1cccc(-c2n[nH]cc2-c2ccc3ncccc3n2)n1]
is provided at a concentration of about 3 μM.

11. The method of claim 8, wherein the cell is a cardiomyocyte.

12. A method for treating heart damage in a subject in need thereof, the method comprising administering to the subject an effective amount of a therapeutic agent selected from: 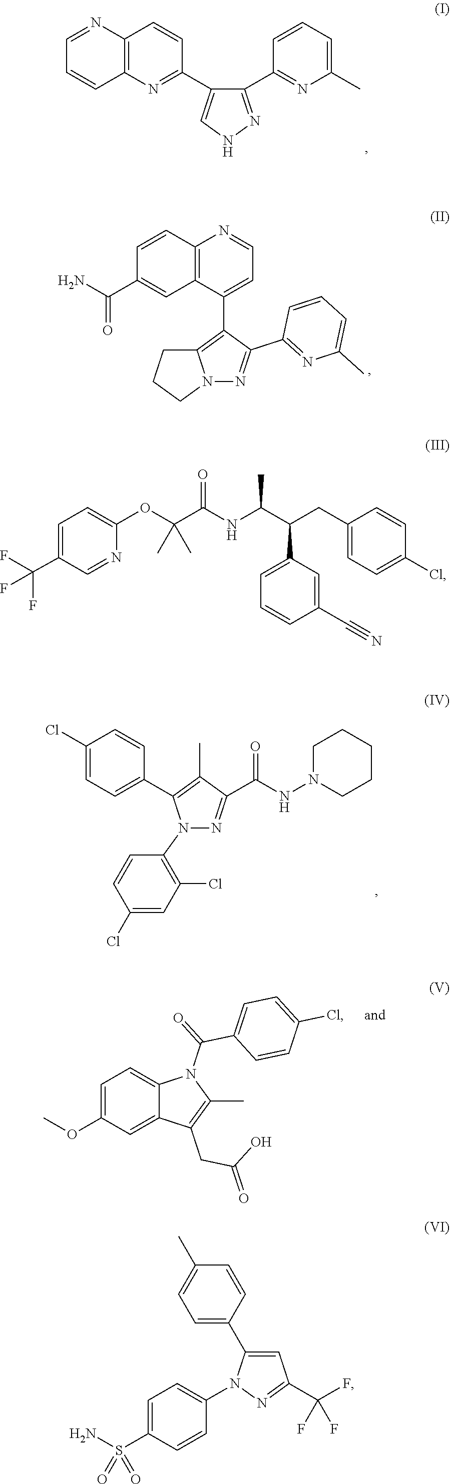
or a pharmaceutically acceptable salt thereof.

13. The method of claim 12, wherein the therapeutic agent is

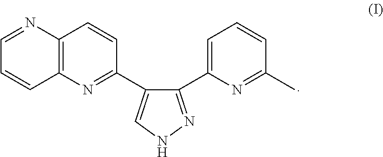 [SMILES: Cc1cccc(-c2n[nH]cc2-c2ccc3ncccc3n2)n1]

14. The method of claim 12, wherein the heart damage is a result of at least one of chemotherapy, a receptor tyrosine kinase inhibitor therapy, recreational drug use, heart disease, and a heart attack.

15. The method of claim 12, further comprising administering an additional therapy selected from chemotherapy, a receptor tyrosine kinase inhibitor therapy, and a heart disease therapy.

16. The method of claim 15, wherein the additional therapy is administered before, during or after the therapeutic agent.

17. The method of claim 12, wherein the subject is a human.

18. A method for treating a subject in need thereof, the method comprising administering to the subject an effective amount of a therapeutic agent selected from: 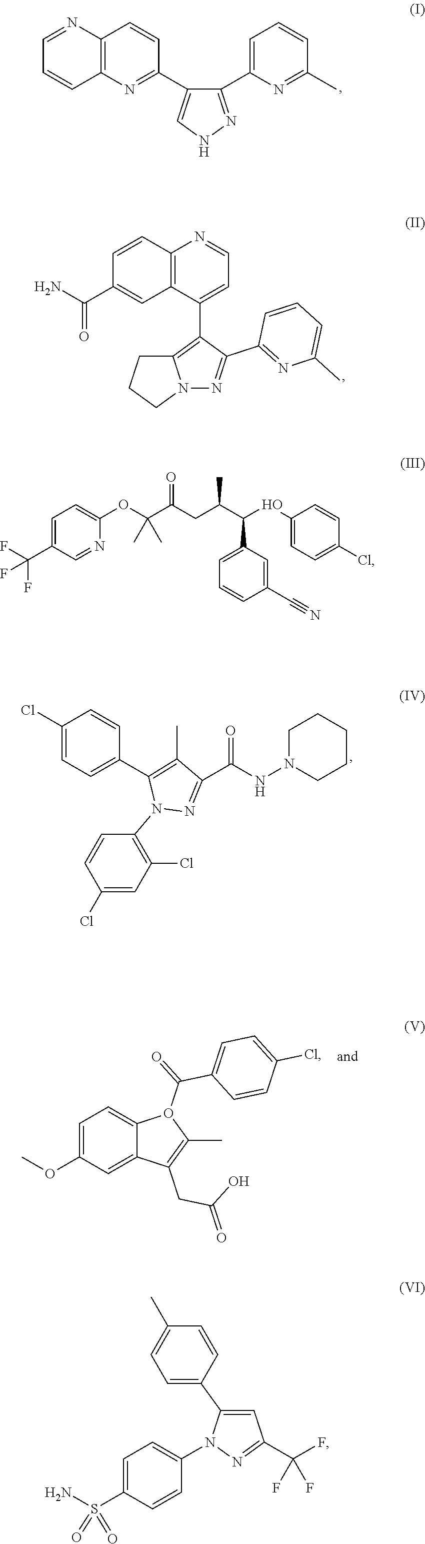
or a pharmaceutically acceptable salt thereof,
wherein the subject is genetically predisposed to a heart defect or a heart condition.

19. The method of claim 18, wherein the subject is genetically predisposed to a heart defect or a heart condition.
